(19) United States
(12) Patent Application Publication
(10) Pub. No.: US 2006/0205949 A1
(43) Pub. Date: Sep. 14, 2006

(54) Crystalline forms of a biphenyl compound

(75) Inventors: Sean M. Dalziel, San Francisco, CA (US); Cristin Hubbard, San Francisco, CA (US); Viengkham Malathong, Irvine, CA (US)

Correspondence Address: THERAVANCE, INC., 901 GATEWAY BOULEVARD, SOUTH SAN FRANCISCO, CA, 94080, US

(73) Assignee: THERAVANCE, INC.

(21) Appl. No.: 11/371,446

(22) Filed: Mar. 9, 2006

Related U.S. Application Data
(60) Provisional application No. 60/660,434, filed on Mar. 10, 2005.

(51) Int. Cl. C07D 401/00

(52) U.S. Cl. 546/209

(57) ABSTRACT

The invention provides crystalline naphthalene-1,5-disulfonic acid salts of biphenyl-2-ylcarbamic acid 1-{2-[5-(4-hydroxybenzylamino)pentylcarbamoyl]ethyl}piperidin-4-yl ester, and pharmaceutically acceptable solvates thereof. The invention also provides pharmaceutical compositions comprising the crystalline compounds or prepared using such compounds; processes and intermediates for preparing the crystalline compounds; and methods of using the compounds to treat pulmonary disorders.

### CROSS-REFERENCE TO RELATED APPLICATIONS

This application claims the benefit of U.S. Provisional Application No. 60/660,434, filed on Mar. 10, 2005; the entire disclosure of which is incorporated herein by reference in its entirety.

### BACKGROUND OF THE INVENTION

1. Field of the Invention

The present invention relates to novel crystalline naphthalene-1,5-disulfonic acid salts of a biphenyl compound and solvates thereof, which are expected to be useful for treating pulmonary disorders. The invention also relates to pharmaceutical compositions comprising the crystalline compounds or prepared from such compounds, processes and intermediates for preparing the crystalline compounds and methods of using such compounds to treat pulmonary disorders.

2. State of the Art

Commonly-assigned U.S. Patent Publication No. 2005/0203083 to Mammen et al. discloses novel biphenyl compounds that are expected to be useful for treating pulmonary disorders such as chronic obstructive pulmonary disease (COPD) and asthma. In particular, the compound biphenyl-2-ylcarbamic acid 1-{2-[5-(4-hydroxybenzylamino)pentyl-carbamoyl]ethyl}piperidin-4-yl ester is specifically disclosed in this application as possessing muscarinic receptor antagonist activity.

The chemical structure of biphenyl-2-ylcarbamic acid 1-{2-[5-(4-hydroxybenzyl amino)pentylcarbamoyl]ethyl}piperidin-4-yl ester is represented by formula I: 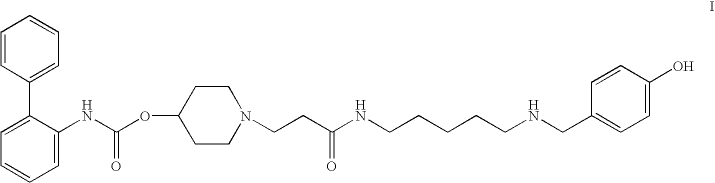
The compound of formula I has been named using the commercially-available AutoNom software (MDL, San Leandro, Calif.).

Therapeutic agents useful for treating pulmonary or respiratory disorders are advantageously administered directly into the respiratory tract by inhalation. In this regard, several types of pharmaceutical inhalation devices have been developed for administering therapeutic agents by inhalation including dry powder inhalers (DPI), metered-dose inhalers (MDI) and nebulizer inhalers. When preparing pharmaceutical compositions and formulations for use in such devices, it is highly desirable to have a crystalline form of the therapeutic agent that is neither hygroscopic nor deliquescent and which has a relatively high melting point thereby allowing the material to be micronized without significant decomposition or loss of crystallinity.

No crystalline salts of the compound of formula I have been reported previously. Accordingly, a need exists for stable, non-deliquescent salt forms of the compound of formula I which have acceptable levels of hygroscopicity and relatively high melting points.

### SUMMARY OF THE INVENTION

The present invention provides crystalline naphthalene-1,5-disulfonic acid salts of biphenyl-2-ylcarbamic acid 1-{2-[5-(4-hydroxybenzylamino)pentylcarbamoyl]ethyl}piperidin-4-yl ester (formula I) and pharmaceutically acceptable solvates thereof. The present invention provides the crystalline compound as a mononapadisylate salt, a heminapadisylate salt or a mononapadisylate ethanolate.

Surprisingly, a crystalline naphthalene-1,5-disulfonic acid form of the compound of formula I has been found not to be deliquescent, even when exposed to up to 90% relative humidity. Additionally, in one embodiment, the crystalline compound of the invention has an acceptable level of hygroscopicity and a high melting point, about 176° C.

Among other uses, a crystalline naphthalene-1,5-disulfonic acid salt of the compound of formula I or a pharmaceutically acceptable solvate thereof, is useful for preparing pharmaceutical compositions expected to have utility for treating pulmonary disorders. Accordingly, one aspect of the invention pertains to a pharmaceutical composition comprising a pharmaceutically acceptable carrier and a therapeutically effective amount of a crystalline naphthalene-1,5-disulfonic acid salt of the compound of formula I or a pharmaceutically acceptable solvate thereof.

Yet another aspect of the invention pertains to compositions comprising a crystalline naphthalene-1,5-disulfonic acid salt of the compound of formula I in combination with one or more other therapeutic agents. Accordingly, in one embodiment, the invention is directed to a composition comprising (a) a pharmaceutically acceptable carrier and a therapeutically effective amount of a crystalline naphthalene-1,5-disulfonic acid salt of the compound of formula I or a pharmaceutically acceptable solvate thereof; and (b) a therapeutically effective amount of an agent selected from a steroidal anti-inflammatory agent such as a corticosteroid; a β₂ adrenergic receptor agonist; a phosphodiesterase-4 inhibitor; or a combination thereof; wherein the salt and the agent are formulated together or separately. When the agent is formulated separately, a pharmaceutically acceptable carrier may be included.

Another aspect of the invention relates to a pharmaceutical composition comprising an aqueous isotonic saline solution comprising a crystalline naphthalene-1,5-disulfonic acid salt of the compound of formula I or a pharmaceutically acceptable solvate thereof, wherein the solution has a pH in the range of from about 4 to 6. In a particular embodiment, an aqueous nebulizer formulation is buffered with citrate buffer to a pH of about 4.5. In another particular embodiment, the aqueous nebulizer formulation contains about 0.1 mg/mL free base equivalents of biphenyl-2-ylcarbamic acid 1-{2-[5-(4-hydroxybenzyl amino)pentylcarbamoyl]ethyl}piperidin-4-yl ester.

In one embodiment, the invention provides a drug delivery device comprising a dry powder inhaler containing a pharmaceutical composition comprising a pharmaceutically acceptable carrier and a crystalline naphthalene-1,5-disulfonic acid salt of the compound of formula I or a pharmaceutically acceptable solvate thereof.

The compound of formula I has muscarinic receptor antagonist activity. Accordingly, a crystalline naphthalene-1,5-disulfonic acid salt of the compound of formula I or a pharmaceutically acceptable solvate thereof, is useful for treating pulmonary disorders such as asthma and chronic obstructive pulmonary disease. Thus, another aspect of the invention pertains to a method for treating a pulmonary disorder comprising administering to a patient a therapeutically effective amount of a crystalline naphthalene-1,5-disulfonic acid salt of the compound of formula I or a pharmaceutically acceptable solvate thereof.

Still another aspect of the invention relates to a method of producing bronchodilation in a patient comprising administering to the patient a bronchodilation-producing amount of a crystalline naphthalene-1,5-disulfonic acid salt of the compound of formula I or a pharmaceutically acceptable solvate thereof. In one embodiment, the compound is administered by inhalation.

The invention also provides a method of treating chronic obstructive pulmonary disease or asthma, comprising administering to a patient a therapeutically effective amount of a crystalline naphthalene-1,5-disulfonic acid salt of the compound of formula I or a pharmaceutically acceptable solvate thereof.

Another aspect of the invention is directed to a method for antagonizing a muscarinic receptor in a mammal comprising administering to the mammal, a therapeutically effective amount of a crystalline naphthalene-1,5-disulfonic acid salt of the compound of formula I or a pharmaceutically acceptable solvate thereof.

The invention is directed to processes for preparing a crystalline naphthalene-1,5-disulfonic acid salt of the compound of formula I or a pharmaceutically acceptable solvate thereof. The invention also provides a process for purifying the compound of formula I comprising forming a crystalline naphthalene-1,5-disulfonic acid salt of biphenyl-2-ylcarbamic acid 1-{2-[5-(4-hydroxybenzylamino)pentylcarbamoyl]ethyl}piperidin-4-yl ester or a pharmaceutically acceptable solvate thereof. The invention is further directed to products prepared by the processes described herein.

The invention is also directed to a crystalline naphthalene-1,5-disulfonic acid salt of the compound of formula I or a pharmaceutically acceptable solvate thereof, in a micronized form; and to pharmaceutical compositions comprising a pharmaceutically acceptable carrier and such micronized crystalline compound.

The invention is also directed to a crystalline naphthalene-1,5-disulfonic acid salt of the compound of formula I or a pharmaceutically acceptable solvate thereof, for use in therapy or as a medicament.

Additionally, the invention relates to the use of a crystalline naphthalene-1,5-disulfonic acid salt of the compound of formula I or a pharmaceutically acceptable solvate thereof, for the manufacture of a medicament; especially for the manufacture of a medicament for the treatment of a pulmonary disorder or for antagonizing a muscarinic receptor in a mammal.

### BRIEF DESCRIPTION OF THE DRAWINGS

Various aspects of the present invention are illustrated by reference to the accompanying drawings.

FIG. 1 shows a powder x-ray diffraction (PXRD) pattern of a crystalline mononapadisylate salt of biphenyl-2-ylcarbamic acid 1-{2-[5-(4-hydroxybenzylamino) pentylcarbamoyl]ethyl}piperidin-4-yl ester (the compound of formula I). FIG. 2 shows a differential scanning calorimetry (DSC) trace for this crystalline salt. FIG. 3 shows a thermal gravimetric analysis (TGA) trace for this crystalline salt. FIG. 4 shows a dynamic moisture sorption (DMS) trace for this crystalline salt. FIG. 5 is a micrographic image of this crystalline salt.

FIG. 6 shows a PXRD pattern of a crystalline heminapadisylate salt of the compound of formula I. This crystalline salt is further characterized by the DSC trace in FIG. 7, the TGA trace in FIG. 8, the DMS trace in FIG. 9, and the micrographic image in FIG. 10.

FIG. 11 shows a PXRD pattern of a crystalline mononapadisylate ethanolate of the compound of formula I. This crystalline solvate is further characterized by the DSC trace in FIG. 12, the TGA trace in FIG. 13, and the micrographic image in FIG. 14.

### DETAILED DESCRIPTION OF THE INVENTION

The invention provides a crystalline naphthalene-1,5-disulfonic acid salt of biphenyl-2-ylcarbamic acid 1-{2-[5-(4-hydroxybenzylamino)pentylcarbamoyl]ethyl}piperidin-4-yl ester or a pharmaceutically acceptable solvate thereof. Naphthalene-1,5-disulfonic acid salts are also sometimes referred to as napadisylate salts. The crystalline compound of the invention can be a mononapadisylate salt, a heminapadisylate salt or a mononapadisylate ethanolate.

### Definitions

When describing the compounds, compositions, methods and processes of the invention, the following terms have the following meanings unless otherwise indicated.

The term “solvate” means a crystal form, where molecules of solvent are incorporated in the unit cell of the crystal lattice, i.e. a naphthalene-1,5-disulfonic acid salt of the compound of formula I, and molecules of a solvent. The solvate may include one or more molecules of solvent, but the number of solvent molecules may also be a fraction of one such as one-half or one-fourth. In the present invention, an exemplary solvate crystal comprises one molecule of ethanol solvent per one molecule of a naphthalene-1,5-disulfonic acid salt of the compound of formula I. Solvates typically have a substantially fixed molar ratio of solute and solvent. This term also includes clathrates, including clathrates with water. Representative solvents include, by way of example, water, methanol, ethanol, isopropanol, acetic acid and the like. When the solvent molecule in a solvate crystal is water, the crystal is called a hydrate, which is one class of solvates.

The term “therapeutically effective amount” means an amount sufficient to effect treatment when administered to a patient in need of treatment. For example, a therapeutically effective amount for antagonizing a muscarinic receptor is that amount which will achieve the desired antagonizing effect. Similarly, a therapeutically effective amount for treating a pulmonary disorder is that amount that will achieve the desired therapeutic result, which may be disease prevention, amelioration, suppression or alleviation, as described below.

The term “treating” or “treatment” as used herein means the treating or treatment of a disease or medical condition (such as COPD) in a patient such as a mammal (particularly a human) that includes:
(a) preventing the disease or medical condition from occurring, i.e., prophylactic treatment of a patient believed to be at risk of contracting or being pre-disposed to such disease or medical condition;
(b) ameliorating the disease or medical condition, i.e., eliminating or causing regression of the disease or medical condition in a patient having such disease or medical condition;
(c) suppressing the disease or medical condition, i.e., slowing or arresting the development of the disease or medical condition in a patient having such disease or medical condition; or
(d) alleviating the symptoms of the disease or medical condition in a patient having such disease or medical condition.

The term “pharmaceutically acceptable” refers to a material that is not biologically or otherwise undesirable. For example, the term “pharmaceutically acceptable carrier” refers to a material that can be incorporated into a composition and administered to a patient without causing undesirable biological effects or interacting in a deleterious manner with other components of the composition. Such pharmaceutically acceptable materials typically have met the required standards of toxicological and manufacturing testing, and include those materials identified as suitable inactive ingredients by the U.S. Food and Drug administration.

The term “unit dosage form” refers to a physically discrete unit suitable for dosing a patient, i.e., each unit containing a predetermined quantity of a compound of the invention calculated to produce the desired therapeutic effect either alone or in combination with one or more additional units. For example, such unit dosage forms may be capsules, tablets, pills, and the like.

### Synthesis

The crystalline compounds of the invention can be synthesized from readily available starting materials as described below and in the Examples. It will be appreciated that while specific process conditions (i.e. reaction temperatures, times, mole ratios of reactants, solvents, pressures, etc.) are given, other process conditions can also be used unless otherwise stated. Generally, the reactions are conducted in a suitable inert diluent, examples of which include, but are not limited to, methanol, ethanol, isopropanol, isobutanol, ethyl acetate, acetonitrile, dichloromethane, methyl t-butyl ether, and the like, and mixtures thereof, optionally containing water. Upon completion of any of the foregoing reactions, the crystalline compounds can be isolated from the reaction mixture by any conventional means such as precipitation, concentration, centrifugation and the like.

The biphenyl-2-ylcarbamic acid 1-{2-[5-(4-hydroxybenzylamino)pentyl carbamoyl]ethyl}piperidin-4-yl ester employed in the invention can be readily prepared from commercially available starting materials and reagents using the procedures described in the Examples, or using the procedures described in U.S. Patent Publication No. 2005/0203083 to Mammen et al.

The molar ratios described in the methods of the invention can be readily determined by various methods available to those skilled in the art. For example, such molar ratios can be readily determined by ¹H NMR. Alternatively, elemental analysis and HPLC methods can be used to determine the molar ratio.

A crystalline mononapadisylate salt of the invention typically contains between about 0.8 and 1.2 molar equivalents of naphthalene-1,5-disulfonic acid per molar equivalent of the compound of formula I, more typically about 1.0 molar equivalents of naphthalene-1,5-disulfonic acid per molar equivalent of the compound of formula I.

A crystalline heminapadisylate salt of the invention typically contains between about 0.35 and 0.65 molar equivalents of naphthalene-1,5-disulfonic acid per molar equivalent of the compound of formula I, more typically about 0.5 molar equivalents of naphthalene-1,5-disulfonic acid per molar equivalent of the compound of formula I.

A crystalline mononapadisylate ethanolate of the invention typically contains about 1.0 molar equivalents of naphthalene-1,5-disulfonic acid and about 1.0 molar equivalents of ethanol per molar equivalent of the compound of formula I.

In general, a crystalline naphthalene-1,5-disulfonic acid salt of the invention can be prepared from biphenyl-2-ylcarbamic acid 1-{2-[5-(4-hydroxybenzylamino) pentylcarbamoyl]ethyl}piperidin-4-yl ester and naphthalene-1,5-disulfonic acid. Suitable inert diluents for this reaction include, but are not limited to, acetonitrile, methanol, ethanol, isopropanol, isobutanol, ethyl acetate and the like, and mixtures thereof, optionally containing water. For example, the ester can be contacted with 1,5 naphthalene disulfonic acid anhydrous, dissolved in acetonitrile and methanol.

Naphthalene-1,5-disulfonic acid (also known as Armstrong's Acid) is commercially available from, for example, Aldrich, Milwaukee, Wis. In one embodiment, the naphthalene-1,5-disulfonic acid employed in preparing the crystalline compounds of the invention is a tetrahydrate. If desired, the naphthalene-1,5-disulfonic acid tetrahydrate employed in the invention can be recrystallized from, for example, acetonitrile prior to use.

There are several methods that can be used to produce the crystalline compounds of the invention. It is noted, however, that the crystalline content as well as the characteristics of the crystals (e.g., shape) may vary, based partly upon the method of preparation, as well as on the solvent composition, and the crystal form. The crystals have been observed as thick rhomboidal plates, as thin diamond shaped plates, needles, and as variations of such.

To prepare a crystalline heminapadisylate salt, the ester is typically dissolved in 3:1 acetonitrile:methanol, diluted with the anhydrous 1,5 naphthalene disulfonic acid dissolved in 3:1 acetonitrile:methanol, and isolated by filtration. Generally, this reaction is conducted in an inert diluent at a temperature ranging from about 0 to 30° C., such as about 22° C. The molar ratio of the ester to 1,5 naphthalene disulfonic acid is about 0.2 to 0.5. In a particular embodiment, 103 g of the ester is taken up in 700 mL of 3:1 acetonitrile:methanol and combined with 1.2 L of 1,5 naphthalene disulfonic acid solution (37.3 g of 1,5 naphthalene disulfonic acid anhydrous in 3.6 L anhydrous in of 3:1 acetonitrile:methanol), and agitated. The slurry is filtered and dried to provide the heminapadisylate salt.

The crystalline mononapadisylate salt can be made from the heminapadisylate salt, by dispersing the heminapadisylate salt in 3:1 acetonitrile:methanol, mixing with 1.0 molar equivalent of the anhydrous 1,5 naphthalene disulfonic acid dissolved in 3:1 acetonitrile:methanol, and isolation by filtration. Generally, this reaction is conducted in an inert diluent at a temperature ranging from about 0 to 30° C., such as about 22° C. In a particular embodiment, 41.5 g of the heminapadisylate salt is taken up in 6 L of 3:1 acetonitrile:methanol and agitated. 1.2 L of 1,5 naphthalene disulfonic acid solution (37.3 g of 1,5 naphthalene disulfonic acid anhydrous in 3.6 L anhydrous in of 3:1 acetonitrile:methanol), 2×, is added, and agitated. The slurry is filtered and dried to provide a mononapadisylate salt.

Alternately, the crystalline mononapadisylate salt can be prepared by contacting the ester with about 1.0 to 1.5 molar equivalents of naphthalene-1,5-disulfonic acid. Generally, this reaction is conducted in an inert diluent at a temperature ranging from about −20 to 65° C., such as about 22° C.

To prepare a crystalline mononapadisylate ethanolate, the ester is typically contacted with about 0.75 to 1.25 molar equivalents of naphthalene-1,5-disulfonic acid. This is solubilized by heating in methanol, then crystallized by adding ethanol to about 3:2 to 9:1 volume ratio, and cooling. Generally, this reaction is conducted in an inert diluent at a temperature ranging from about −20 to 65° C., such as about 22° C.

### Crystalline Properties

Among other advantages, it has been discovered that forming a crystalline naphthalene-1,5-disulfonic acid salt of the compound of formula I or solvate thereof, is useful for purifying the compound of formula I. For example, a crystalline naphthalene-1,5-disulfonic acid mononapadisylate salt of the invention has a purity greater than 98%, and typically greater than 99.5%.

As is well known in the field of powder x-ray diffraction, relative peak heights of PXRD spectra are dependent on a number of factors relating to sample preparation and instrument geometry, while peak positions are relatively insensitive to experimental details. Thus, in one embodiment, the crystalline compounds of the invention are characterized by a PXRD pattern having certain peak positions.

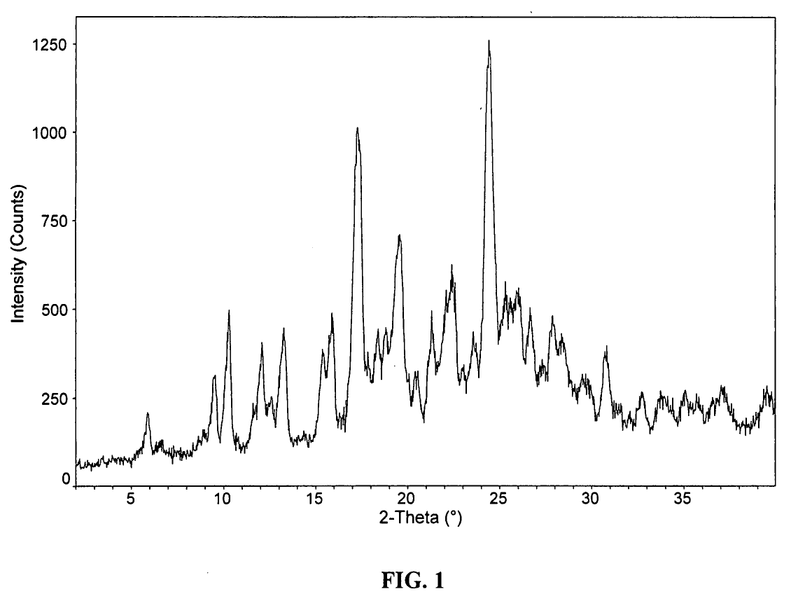

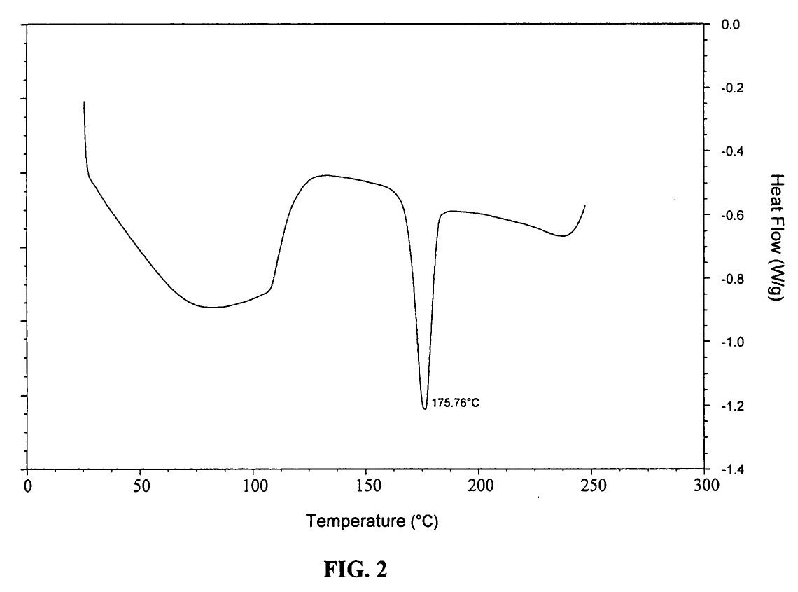

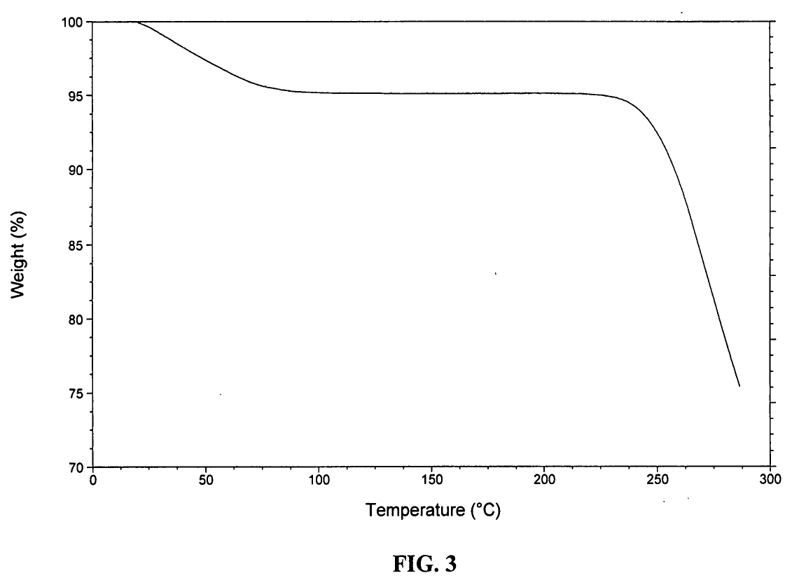

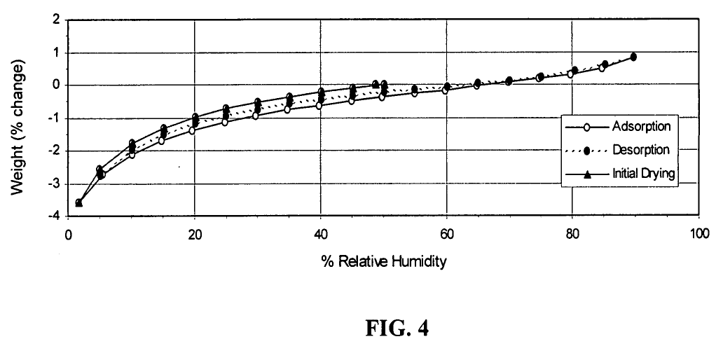

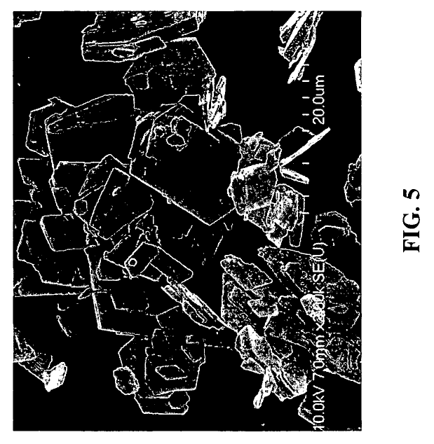

A crystalline mononapadisylate salt of the compound of formula I is characterized by a PXRD pattern in which the peak positions are substantially in accordance with those shown in FIG. 1. Those peaks are listed below, in order of descending relative intensity.

| 2-Theta (°) | Height | I % |
|---|---|---|
| 24.41 | 858 | 100 |
| 17.30 | 770 | 89.7 |
| 19.58 | 421 | 49.1 |
| 10.31 | 362 | 42.2 |
| 15.89 | 320 | 37.3 |
| 22.40 | 303 | 35.3 |
| 13.28 | 282 | 32.9 |
| 12.08 | 246 | 28.7 |
| 15.38 | 232 | 27 |
| 30.83 | 195 | 22.7 |
| 21.32 | 191 | 22.3 |
| 26.03 | 184 | 21.4 |
| 9.55 | 173 | 20.2 |
| 27.89 | 173 | 20.2 |
| 26.66 | 164 | 19.1 |
| 25.31 | 143 | 16.7 |
| 28.37 | 129 | 15 |
| 5.90 | 128 | 14.9 |
| 23.57 | 109 | 12.7 |
| 18.40 | 108 | 12.6 |

Thus, in one embodiment, the crystalline mononapadisylate salt of the present invention is characterized by a powder x-ray diffraction (PXRD) pattern having two or more diffraction peaks at 20 values selected from 5.90±0.2, 9.55±0.2, 10.31±0.2, 12.08±0.2, 13.28±0.2, 15.38±0.2, 15.89±0.2, 17.30±0.2, 18.40±0.2, 19.58±0.2, 21.32±0.2, 22.40±0.2, 23.57±0.2, 24.41±0.2, 25.31±0.2, 26.03±0.2, 26.66±0.2, 27.89±0.2, 28.37±0.2, and 30.83±0.2. In particular, in this embodiment, the crystalline form is characterized by a powder x-ray diffraction pattern comprising diffraction peaks at 20 values of 10.31±0.2, 17.30±0.2, 19.58±0.2, and 24.41±0.2.

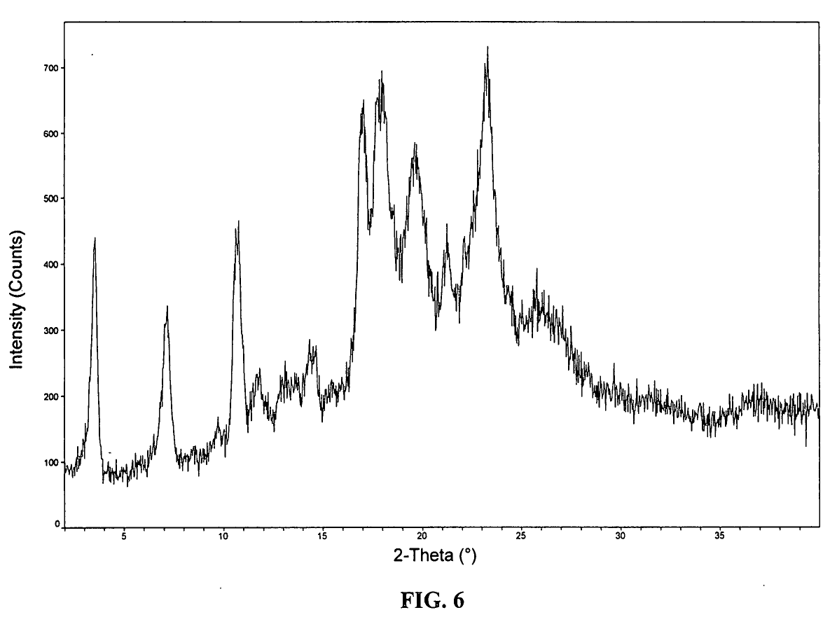

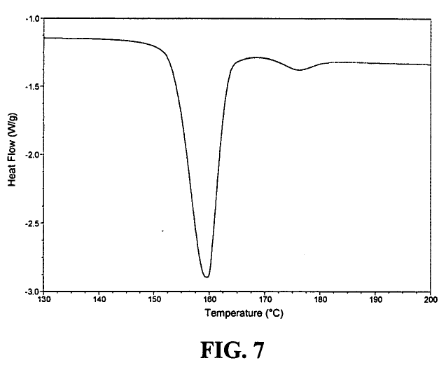

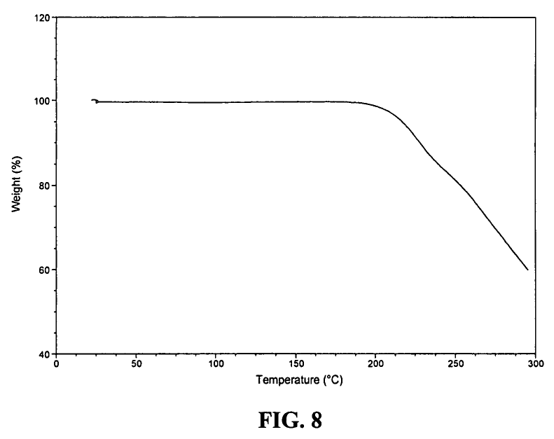

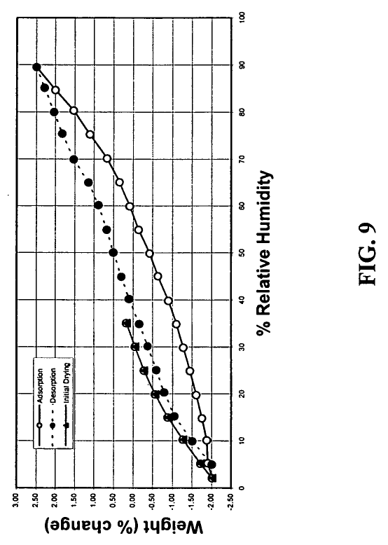

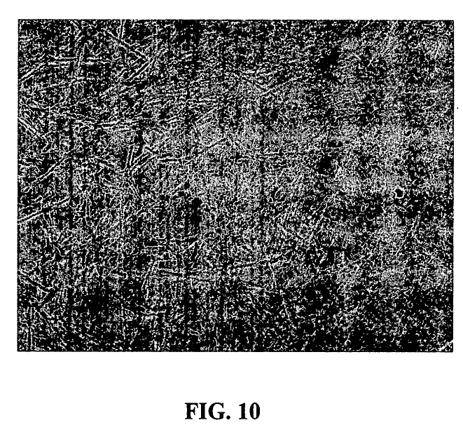

A crystalline heminapadisylate salt of the compound of formula I is characterized by a PXRD pattern in which the peak positions are substantially in accordance with those shown in FIG. 6. Those peaks are listed below, in order of descending relative intensity.

| 2-Theta (°) | Height | I % |
|---|---|---|
| 17.06 | 442 | 100 |
| 3.56 | 351 | 79.4 |
| 23.30 | 346 | 78.3 |
| 10.76 | 308 | 69.7 |
| 7.19 | 240 | 54.3 |
| 17.84 | 233 | 52.7 |
| 19.61 | 189 | 42.8 |
| 19.94 | 132 | 29.9 |
| 21.23 | 115 | 26 |
| 25.78 | 89 | 20.1 |
| 14.65 | 88 | 19.9 |
| 11.81 | 74 | 16.7 |
| 13.11 | 73 | 16.5 |
| 9.71 | 40 | 9 |

Thus, in one embodiment, the crystalline heminapadisylate salt of the present invention is characterized by a powder x-ray diffraction (PXRD) pattern having two or more diffraction peaks at 20 values selected from 3.56±0.2, 7.19±0.2, 9.71±0.2, 10.76±0.2, 11.81±0.2, 13.11±0.2, 14.65±0.2, 17.06±0.2, 17.84±0.2, 19.61±0.2, 19.94±0.2, 21.23±0.2, 23.30±0.2, and 25.78±0.2. In particular, in this embodiment, the crystalline form is characterized by a powder x-ray diffraction pattern comprising diffraction peaks at 20 values of 3.56±0.2, 10.76±0.2, 17.06±0.2, and 23.30±0.2.

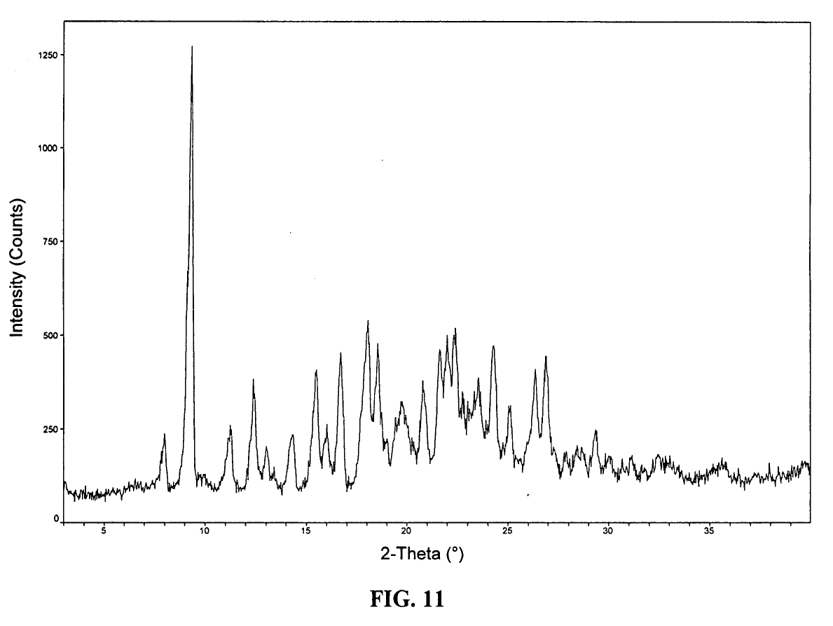

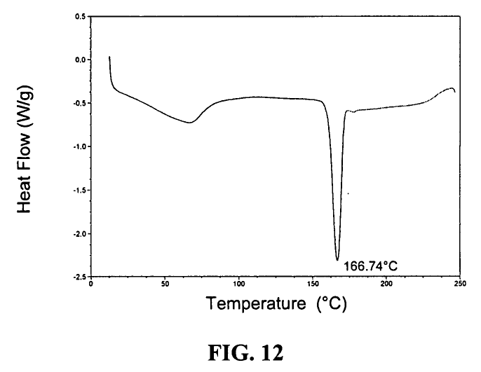

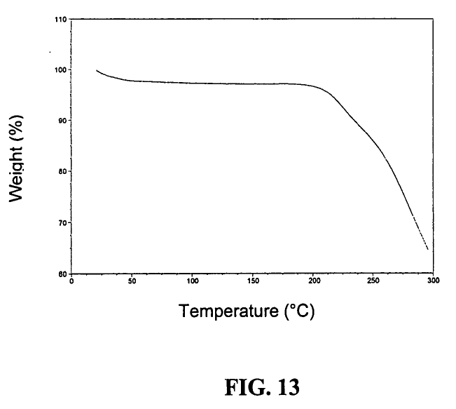

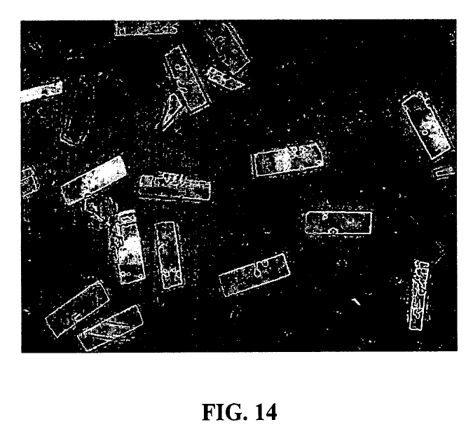

A crystalline mononapadisylate ethanolate of the compound of formula I is characterized by a PXRD pattern in which the peak positions are substantially in accordance with those shown in FIG. 11. Those peaks are listed below, in order of descending relative intensity.

| 2-Theta (°) | Height | I % |
|---|---|---|
| 9.36 | 1182 | 100 |
| 18.06 | 440 | 37.2 |
| 16.71 | 326 | 27.6 |
| 18.54 | 314 | 26.6 |
| 15.51 | 290 | 24.5 |
| 26.91 | 285 | 24.1 |
| 21.99 | 275 | 23.3 |
| 12.40 | 271 | 22.9 |
| 22.41 | 263 | 22.3 |
| 24.30 | 249 | 21.1 |
| 21.63 | 233 | 19.7 |
| 26.37 | 233 | 19.7 |
| 20.79 | 196 | 16.6 |
| 11.25 | 160 | 13.5 |
| 8.01 | 148 | 12.5 |
| 19.77 | 147 | 12.4 |
| 16.02 | 143 | 12.1 |
| 14.31 | 140 | 11.8 |
| 23.55 | 135 | 11.4 |
| 25.13 | 135 | 11.4 |

Thus, in one embodiment, the crystalline mononapadisylate ethanolate of the present invention is characterized by a powder x-ray diffraction (PXRD) pattern having two or more diffraction peaks at 20 values selected from 8.01±0.2, 9.36±0.2, 11.25±0.2, 12.40±0.2, 14.31±0.2, 15.51±0.2, 16.02±0.2, 16.71±0.2, 18.06±0.2, 18.54±0.2, 19.77±0.2, 20.79±0.2, 21.63±0.2, 21.99±0.2, 22.41±0.2, 23.55±0.2, 24.30±0.2, 25.13±0.2, 26.37±0.2, and 26.91±0.2. In particular, in this embodiment, the crystalline form is characterized by a powder x-ray diffraction pattern comprising diffraction peaks at 20 values of 9.36±0.2, 16.71±0.2, 18.06±0.2, and 18.54±0.2.

In yet another embodiment, a crystalline mononapadisylate salt of the compound of formula I is characterized by its differential scanning calorimetry (DSC) trace which shows a peak endothermic heat flow at about 176° C. as illustrated in FIG. 2; a crystalline heminapadisylate of the compound of formula I is characterized by its differential scanning calorimetry (DSC) trace which shows a peak endothermic heat flow at about 160° C., as illustrated in FIG. 7; and a crystalline mononapadisylate ethanolate of the compound of formula I is characterized by its differential scanning calorimetry (DSC) trace which shows a peak endothermic heat flow at about 167° C., as illustrated in FIG. 12.

The crystalline compounds of the invention have been demonstrated to have a reversible sorption/desorption profile with an acceptable, moderate level of hygroscopicity. In particular, a crystalline mononapadisylate salt of the compound of formula I has been demonstrated to exhibit less than 0.8% weight gain when exposed to the critical range of 40-75% relative humidity.

Additionally, the crystalline compounds of the invention have been found to be stable upon exposure to elevated temperature and humidity. For example, after storage for 6 weeks at 40° C. and 75% relative humidity, analysis of such crystalline compounds by high performance liquid chromatography (HPLC) showed no detectable chemical degradation (i.e., less than 0.5% degradation).

These properties of the crystalline compounds of the invention are further illustrated in the Examples below.

### Pharmaceutical Compositions and Formulations

A naphthalene-1,5-disulfonic acid salt of the compound of formula I or solvate thereof, is typically administered to a patient in the form of a pharmaceutical composition or formulation. Such pharmaceutical compositions may be administered to the patient by any acceptable route of administration including, but not limited to, inhaled, oral, nasal, topical (including transdermal) and parenteral modes of administration. However, it will be understood by those skilled in the art that, once the crystalline compound of the invention has been formulated, it may no longer be in crystalline form, e.g., the compound may be dissolved in a suitable carrier.

Accordingly, in one embodiment, the invention is directed to a pharmaceutical composition comprising a pharmaceutically acceptable carrier or excipient and a naphthalene-1,5-disulfonic acid salt of biphenyl-2-ylcarbamic acid 1-{2-[5-(4-hydroxybenzylamino)pentylcarbamoyl]ethyl}piperidin-4-yl ester or solvate thereof. The pharmaceutical composition may contain other therapeutic and/or formulating agents if desired.

The pharmaceutical compositions of the invention typically contain a therapeutically effective amount of a naphthalene-1,5-disulfonic acid salt of biphenyl-2-ylcarbamic acid 1-{2-[5-(4-hydroxybenzylamino)pentylcarbamoyl]ethyl}piperidin-4-yl ester or solvate thereof, as the active agent. Typically, the pharmaceutical composition will contain from about 0.01 to 95% by weight of the active agent; including, from about 0.01 to 30% by weight; such as from about 0.01 to 10% by weight of the active agent.

Any conventional carrier or excipient may be used in the pharmaceutical compositions of the invention. The choice of a particular carrier or excipient, or combination of carriers or excipients, will depend on the mode of administration being used to treat a particular patient or type of medical condition or disease state. In this regard, the preparation of a suitable pharmaceutical composition for a particular mode of administration is well within the scope of those skilled in the pharmaceutical arts. Additionally, the ingredients for such compositions are commercially available from, for example, Sigma, P.O. Box 14508, St. Louis, Mo. 63178. By way of further illustration, conventional formulation techniques are described in Remington: The Science and Practice of Pharmacy, 20^{th} Edition, Lippincott Williams & White, Baltimore, Md. (2000); and H. C. Ansel et al., Pharmaceutical Dosage Forms and Drug Delivery Systems, 7^{th} Edition, Lippincott Williams & White, Baltimore, Md. (1999).

Representative examples of materials that can serve as pharmaceutically acceptable carriers include, but are not limited to, the following: sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; phosphate buffer solutions; compressed propellant gases such as chlorofluorocarbons and hydrofluorocarbons; and other non-toxic compatible substances employed in pharmaceutical compositions.

The pharmaceutical compositions of the invention are typically prepared by thoroughly and intimately mixing or blending a crystalline compound of the invention with a pharmaceutically acceptable carrier and one or more optional ingredients. If necessary or desired, the resulting uniformly blended mixture can then be shaped or loaded into tablets, capsules, pills, canisters, cartridges, dispensers and the like using conventional procedures and equipment.

In one embodiment, the pharmaceutical compositions of the invention are suitable for inhaled administration. Suitable pharmaceutical compositions for inhaled administration will typically be in the form of an aerosol or a powder. Such compositions are generally administered using well-known delivery devices such as a nebulizer inhaler, a metered-dose inhaler (MDI), a dry powder inhaler (DPI) or a similar delivery device.

In a specific embodiment of the invention, the pharmaceutical composition comprising the active agent is administered by inhalation using a nebulizer inhaler. Such nebulizer devices typically produce a stream of high velocity air that causes the pharmaceutical composition comprising the active agent to spray as a mist that is carried into the patient's respiratory tract. Accordingly, when formulated for use in a nebulizer inhaler, the active agent is typically dissolved in a suitable carrier to form a solution. Suitable nebulizer devices are commercially available, for example, by PARI GmbH (Starnberg, German). Other nebulizer devices include Respimat (Boehringer Ingelheim) and those described, for example, in U.S. Pat. No. 6,123,068 to Lloyd et al. and WO 97/12687 (Eicher et al.), the disclosures of which are incorporated herein by reference in their entirety.

A representative pharmaceutical composition for use in a nebulizer inhaler comprises an aqueous solution comprising from about 0.05 μg/mL to 10 mg/mL of a naphthalene-1,5-disulfonic acid salt of compound of formula I or a pharmaceutically acceptable solvate thereof. In one embodiment, the aqueous nebulizer formulation is isotonic. In one embodiment, the aqueous nebulizer formulation has a pH in the range of from about 4 to 6. In a particular embodiment, the aqueous nebulizer formulation is buffered with citrate buffer to a pH of about 5. In another particular embodiment, the aqueous nebulizer formulation contains from about 0.1 mg/mL to 1.0 mg/mL free base equivalents of biphenyl-2-ylcarbamic acid 1-{2-[5-(4-hydroxybenzylamino) pentylcarbamoyl]ethyl}piperidin-4-yl ester.

In another specific embodiment of the invention, the pharmaceutical composition comprising the active agent is administered by inhalation using a DPI. Such DPIs typically administer the active agent as a free-flowing powder that is dispersed in a patient's air-stream during inspiration. In order to achieve a free flowing powder, the active agent is typically formulated with a suitable excipient such as lactose or starch. Micronization is a common method of reducing crystal size to that suitable for pulmonary delivery. Typically, the active agent is micronized and combined with a suitable carrier to form a suspension of micronized particles of respirable size, where “micronized particles” or “micronized form” means at least about 90% of the particles have a diameter of less than about 10 μm. Other methods of reducing particle size may also be used such as fine milling, chopping, crushing, grinding, milling, screening, trituration, pulverization, and so forth, as long as the desired particle size can be obtained.

A representative pharmaceutical composition for use in a DPI comprises dry lactose having a particle size between about 1 μm and 100 μm and micronized particles of a naphthalene-1,5-disulfonic acid salt of compound of formula I or a pharmaceutically acceptable solvate thereof. Such a dry powder formulation can be made, for example, by combining the lactose with the active agent and then dry blending the components. Alternatively, if desired, the active agent can be formulated without an excipient. The pharmaceutical composition is then typically loaded into a dry powder dispenser, or into inhalation cartridges or capsules for use with a dry powder delivery device.

Examples of DPI delivery devices include Diskhaler (GlaxoSmithKline, Research Triangle Park, N.C.; see, e.g., U.S. Pat. No. 5,035,237 to Newell et al.); Diskus (GlaxoSmithKline; see, e.g., U.S. Pat. No. 6,378,519 to Davies et al.); Turbuhaler (AstraZeneca, Wilmington, Del.; see, e.g., U.S. Pat. No. 4,524,769 to Wetterlin); Rotahaler (GlaxoSmithKline; see, e.g., U.S. Pat. No. 4,353,365 to Hallworth et al.) and Handihaler (Boehringer Ingelheim). Further examples of suitable DPI devices are described in U.S. Pat. No. 5,415,162 to Casper et al., U.S. Pat. No. 5,239,993 to Evans, and U.S. Pat. No. 5,715,810 to Armstrong et al., and references cited therein. The disclosures of the aforementioned patents are incorporated herein by reference in their entirety.

In yet another specific embodiment of the invention, the pharmaceutical composition comprising the active agent is administered by inhalation using an MDI, which typically discharges a measured amount of the active agent using compressed propellant gas. Accordingly, pharmaceutical compositions administered using an MDI typically comprise a solution or suspension of the active agent in a liquefied propellant. Any suitable liquefied propellant may be employed including chlorofluorocarbons such as CCl₃F, and hydrofluoroalkanes (HFAs) such as 1,1,1,2-tetrafluoroethane (HFA 134a) and 1,1,1,2,3,3,3-heptafluoro-n-propane, (HFA 227ea). Due to concerns about chlorofluorocarbons affecting the ozone layer, formulations containing HFAs are generally preferred. Additional optional components of HFA formulations include co-solvents such as ethanol or pentane, and surfactants such as sorbitan trioleate, oleic acid, lecithin, and glycerin. See, for example, U.S. Pat. No. 5,225,183 to Purewal et al., EP 0717987 A2 (Minnesota Mining and Manufacturing Company), and WO 92/22286 (Minnesota Mining and Manufacturing Company), the disclosures of which are incorporated herein by reference in their entirety.

A representative pharmaceutical composition for use in an MDI comprises from about 0.01 to 5% by weight of a naphthalene-1,5-disulfonic acid salt of compound of formula I, or a pharmaceutically acceptable solvate thereof; from about 0 to 20% by weight ethanol; and from about 0 to 5% by weight surfactant; with the remainder being an HFA propellant.

Such compositions are typically prepared by adding chilled or pressurized hydrofluoroalkane to a suitable container containing the active agent, ethanol (if present) and the surfactant (if present). To prepare a suspension, the active agent is micronized and then combined with the propellant. The formulation is then loaded into an aerosol canister, which forms a portion of a metered-dose inhaler device. Examples of MDI devices developed specifically for use with HFA propellants are described in U.S. Pat. No. 6,006,745 to Marecki and U.S. Pat. No. 6,143,277 to Ashurst et al. Alternatively, a suspension formulation can be prepared by spray drying a coating of surfactant on micronized particles of the active agent. See, for example, WO 99/53901 (Glaxo Group Ltd.) and WO 00/61108 (Glaxo Group Ltd.). The disclosures of the aforementioned patents and publications are incorporated herein by reference in their entirety.

For additional examples of processes of preparing respirable particles, and formulations and devices suitable for inhalation dosing see U.S. Pat. No. 6,268,533 to Gao et al.; U.S. Pat. No. 5,983,956 to Trofast; U.S. Pat. No. 5,874,063 to Briggner et al.; and U.S. Pat. No. 6,221,398 to Jakupovic et al.; and WO 99/55319 (Glaxo Group Ltd.) and WO 00/30614 (AstraZeneca AB); the disclosures of which are incorporated herein by reference in their entirety.

In another embodiment, the pharmaceutical compositions of the invention are suitable for oral administration. Suitable pharmaceutical compositions for oral administration may be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of a salt of the invention as an active ingredient. The pharmaceutical composition may be packaged in a unit dosage form.

When intended for oral administration in a solid dosage form (i.e., as capsules, tablets, pills and the like), the pharmaceutical compositions of the invention will typically comprise a crystalline compound of the present invention as the active ingredient and one or more pharmaceutically acceptable carriers such as sodium citrate or dicalcium phosphate. Optionally or alternatively, such solid dosage forms may also comprise: fillers or extenders such as starches, lactose, sucrose, glucose, mannitol, and/or silicic acid; binders such as carboxymethylcellulose, alginates, gelatin, polyvinyl pyrrolidone, sucrose and/or acacia; humectants such as glycerol; disintegrating agents such as agar-agar, calcium carbonate, potato or tapioca starch, alginic acid, certain silicates, and/or sodium carbonate; solution retarding agents such as paraffin; absorption accelerators such as quaternary ammonium compounds; wetting agents such as cetyl alcohol and/or glycerol monostearate; absorbents such as kaolin and/or bentonite clay; lubricants such as talc, calcium stearate, magnesium stearate, solid polyethylene glycols, sodium lauryl sulfate, and/or mixtures thereof; coloring agents; and buffering agents.

Release agents, wetting agents, coating agents, sweetening, flavoring and perfuming agents, preservatives and antioxidants can also be present in the pharmaceutical compositions of the invention. Examples of pharmaceutically acceptable antioxidants include: water-soluble antioxidants such as ascorbic acid, cysteine hydrochloride, sodium bisulfate, sodium metabisulfate sodium sulfite and the like; oil-soluble antioxidants such as ascorbyl palmitate, butylated hydroxyanisole (BHA), butylated hydroxytoluene (BHT), lecithin, propyl gallate, alpha-tocopherol, and the like; and metal-chelating agents such as citric acid, ethylenediamine tetraacetic acid (EDTA), sorbitol, tartaric acid, phosphoric acid, and the like. Coating agents for tablets, capsules, pills and like, include those used for enteric coatings such as cellulose acetate phthalate (CAP), polyvinyl acetate phthalate (PVAP), hydroxypropyl methylcellulose phthalate, methacrylic acid-methacrylic acid ester copolymers, cellulose acetate trimellitate (CAT), carboxymethyl ethyl cellulose (CMEC), hydroxypropyl methyl cellulose acetate succinate (HPMCAS), and the like.

If desired, the pharmaceutical compositions of the invention may also be formulated to provide slow or controlled release of the active ingredient using, by way of example, hydroxypropyl methyl cellulose in varying proportions; or other polymer matrices, liposomes and/or microspheres.

In addition, the pharmaceutical compositions of the invention may optionally contain opacifying agents and may be formulated so that they release the active ingredient only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Suitable liquid dosage forms for oral administration include, by way of illustration, pharmaceutically acceptable emulsions, microemulsions, solutions, suspensions, syrups and elixirs. Such liquid dosage forms typically comprise the active ingredient and an inert diluent such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, oils (esp., cottonseed, groundnut, corn, germ, olive, castor and sesame oils), glycerol, tetrahydrofuryl alcohol, polyethylene glycols and fatty acid esters of sorbitan, and mixtures thereof. Suspensions, in addition to the active ingredient, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The crystalline compounds of the invention can also be administered transdermally using known transdermal delivery systems and excipients. For example, a compound of the invention can be admixed with permeation enhancers such as propylene glycol, polyethylene glycol monolaurate, azacycloalkan-2-ones and the like, and incorporated into a patch or similar delivery system. Additional excipients including gelling agents, emulsifiers and buffers, may be used in such transdermal compositions if desired.

The crystalline compounds of the invention can also be co-administered with other therapeutic agents. This combination therapy involves using a compound of the invention combined with one or more of these secondary agents, either formulated together (e.g., packaged together in a single formulation) or formulated separately (e.g., packaged as separate unit dosage forms). Methods of formulating multiple agents together in the same formulation or in separate unit dosage forms, are well known in the art.

The additional therapeutic agent(s) can be selected from other bronchodilators (e.g., PDE₃ inhibitors, adenosine 2b modulators and β₂ adrenergic receptor agonists); anti-inflammatory agents (e.g., steroidal anti-inflammatory agents such as corticosteroids; non-steroidal anti-inflammatory agents (NSA/Ds), and PDE₄ inhibitors); other muscarinic receptor antagonists (i.e., antichlolinergic agents); antiinfective agents (e.g., Gram positive and Gram negative antibiotics or antivirals); antihistamines; protease inhibitors; and afferent blockers (e.g., D₂ agonists and neurokinin modulators).

One particular embodiment of the invention is directed to a composition comprising (a) a pharmaceutically acceptable carrier and a therapeutically effective amount of a crystalline naphthalene-1,5-disulfonic acid salt of the compound of formula I or a pharmaceutically acceptable solvate thereof; and (b) a pharmaceutically acceptable carrier and a therapeutically effective amount of an agent selected from a steroidal anti-inflammatory agent such as a corticosteroid; a β₂ adrenergic receptor agonist; a phosphodiesterase-4 inhibitor; or a combination thereof; wherein the compound of formula I and the agent are formulated together or separately. In another embodiment, (b) is a pharmaceutically acceptable carrier and a therapeutically effective amount of a β₂ adrenergic receptor agonist and a steroidal anti-inflammatory agent. The secondary agents can be used in the form of pharmaceutically acceptable salts or solvates, and if appropriate, as optically pure stereoisomers.

Representative β₂ adrenergic receptor agonists that can be used in combination with crystalline compounds of the invention include, but are not limited to, salmeterol, salbutamol, formoterol, salmefamol, fenoterol, terbutaline, albuterol, isoetharine, metaproterenol, bitolterol, pirbuterol, levalbuterol and the like, or pharmaceutically acceptable salts thereof. Other β₂ adrenergic receptor agonists that can be used include, but are not limited to, 3-(4-{[6-({(2R)-2-hydroxy-2-[4-hydroxy-3-(hydroxymethyl)-phenyl]ethyl}amino)-hexyl]oxy}butyl)benzenesulfonamide and 3-(−3-{[7-({(2R)-2-hydroxy-2-[4-hydroxy-3-(hydroxymethyl)phenyl]ethyl}-amino)heptyl]oxy}-propyl)benzenesulfonamide and related compounds described in WO 02/066422 (Glaxo Group Ltd.); 3-[3-(4-{[6-([(2R)-2-hydroxy-2-[4-hydroxy-3-(hydroxymethyl)phenyl]ethyl}amino)hexyl]oxy}butyl) phenyl]imidazolidine-2,4-dione and related compounds described in WO 02/070490 (Glaxo Group Ltd.); 3-(4-{[6-({(2R)-2-[3-(formylamino)-4-hydroxyphenyl]-2-hydroxyethyl}amino)hexyl]oxy}butyl)-benzenesulfonamide, 3-(4-{[6-({(2S)-2-[3-(formylamino)-4-hydroxyphenyl]-2-hydroxyethyl}amino)hexyl]oxy}butyl)-benzenesulfonamide, 3-(4-{[6-({(2R/S)-2-[3-(formylamino)-4-hydroxyphenyl]-2-hydroxyethyl}amino)hexyl]oxy}butyl)-benzenesulfonamide, N-(tert-butyl)-3-(4-{[6-({(2R)-2-[3-(formylamino)-4-hydroxyphenyl]-2-hydroxyethyl}amino)hexyl]-oxy}butyl) benzenesulfonamide, N-(tert-butyl)-3-(4-{[6-({(2S)-2-[3-(formylamino)-4-hydroxyphenyl]-2-hydroxyethyl}amino)-hexyl]oxy}butyl)-benzenesulfonamide, N-(tert-butyl)-3-(4-{[6-({(2R/S)-2-[3-(formylamino)-4-hydroxyphenyl]-2-hydroxyethyl}amino)hexyl]-oxy}butyl) benzenesulfonamide and related compounds described in WO 02/076933 (Glaxo Group Ltd.); 4-{(1R)-2-[(6-{2-[(2,6-dichlorobenzyl)oxy]ethoxy}hexyl)amino]-1-hydroxyethyl}-2-(hydroxymethyl)phenol and related compounds described in WO 03/024439 (Glaxo Group Ltd.); N-{2-[4-((R)-2-hydroxy-2-phenylethylamino)phenyl]ethyl}-(R)-2-hydroxy-2-(3-formamido-4-hydroxyphenyl)ethylamine and related compounds described in U.S. Pat. No. 6,576,793 to Moran et al.; N-{2-[4-(3-phenyl-4-methoxyphenyl)aminophenyl]ethyl}-(R)-2-hydroxy-2-(8-hydroxy-2(1H)-quinolinon-5-yl)ethylamine and related compounds described in U.S. Pat. No. 6,653,323 to Moran et al.; and pharmaceutically acceptable salts thereof. In a particular embodiment, the β₂-adrenoreceptor agonist is a crystalline monohydrochloride salt of N-{2-[4-((R)-2-hydroxy-2-phenylethylamino)phenyl]ethyl}-(R)-2-hydroxy-2-(3-formamido-4-hydroxyphenyl)ethylamine. When employed, the β2-adrenoreceptor agonist will be present in the pharmaceutical composition in a therapeutically effective amount. Typically, the β₂-adrenoreceptor agonist will be present in an amount sufficient to provide from about 0.05 μg to 500 μg per dose. The disclosures of the aforementioned patents and publications are incorporated herein by reference in their entirety.

Representative steroidal anti-inflammatory agents that can be used in combination with crystalline compounds of the invention include, but are not limited to, methyl prednisolone, prednisolone, dexamethasone, fluticasone propionate, 6α,9α-difluoro-17α-[(2-furanyl carbonyl)oxy]-11β-hydroxy-16α-methyl-3-oxoandrosta-1,4-diene-17α-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydrofuran-3 S-yl) ester, beclomethasone esters (e.g., the 17-propionate ester or the 17,21-dipropionate ester), budesonide, flunisolide, mometasone esters (e.g., the furoate ester), triamcinolone acetonide, rofleponide, ciclesonide, butixocort propionate, RPR-106541, ST-126 and the like, or pharmaceutically-acceptable salts thereof. When employed, the steroidal anti-inflammatory agent will be present in the composition in a therapeutically effective amount. Typically, the steroidal anti-inflammatory agent will be present in an amount sufficient to provide from about 0.05 μg to 500 μg per dose.

An exemplary combination is a naphthalene-1,5-disulfonic acid salt of biphenyl-2-ylcarbamic acid 1-{2-[5-(4-hydroxybenzylamino)pentylcarbamoyl]ethyl}piperidin-4-yl ester or solvate thereof, co-administered with salmeterol as the β₂ adrenergic receptor agonist, and fluticasone propionate as the steroidal anti-inflammatory agent. Another exemplary combination is a naphthalene-1,5-disulfonic acid salt of biphenyl-2-ylcarbamic acid 1-{2-[5-(4-hydroxybenzylamino)pentylcarbamoyl]ethyl}piperidin-4-yl ester or solvate thereof, co-administered with a crystalline monohydrochloride salt of N-{2-[4-((R)-2-hydroxy-2-phenylethylamino)phenyl]ethyl}-(R)-2-hydroxy-2-(3-formamido-4-hydroxyphenyl)ethylamine as the β₂-adrenoreceptor agonist, and 6α,9α-difluoro-17α-[(2-furanylcarbonyl)oxy]-11β-hydroxy-16α-methyl-3-oxoandrosta-1,4-diene-17β-carbothioic acid S-fluoromethyl ester as the steroidal anti-inflammatory agent. As noted above, these agents can be formulated together or separately.

Other suitable combinations include, for example, other anti-inflammatory agents, e.g., NSAIDs (e.g., sodium cromoglycate, nedocromil sodium, and phosphodiesterase (PDE) inhibitors such as theophylline, PDE4 inhibitors and mixed PDE3/PDE4 inhibitors); leukotriene antagonists (e.g., monteleukast); inhibitors of leukotriene synthesis; iNOS inhibitors; protease inhibitors such as tryptase and elastase inhibitors; beta-2 integrin antagonists and adenosine receptor agonists or antagonists (e.g., adenosine 2a agonists); cytokine antagonists (e.g., chemokine antagonists such as, an interleukin antibody (αIL antibody), specifically, an αIL-4 therapy, an αIL-13 therapy, or a combination thereof); or inhibitors of cytokine synthesis.

Representative phosphodiesterase-4 (PDE4) inhibitors or mixed PDE3/PDE4 inhibitors that can be used in combination with the crystalline compounds of the invention include, but are not limited to cis 4-cyano-4-(3-cyclopentyloxy-4-methoxyphenyl) cyclohexan-1-carboxylic acid, 2-carbomethoxy-4-cyano-4-(3-cyclopropylmethoxy-4-difluoromethoxyphenyl)cyclohexan-1-one; cis-[4-cyano-4-(3-cyclopropylmethoxy-4-difluoromethoxyphenyl)cyclohexan-1-ol]; cis-4-cyano-4-[3-(cyclopentyloxy)-4-methoxyphenyl]cyclohexane-1-carboxylic acid and the like, or pharmaceutically acceptable salts thereof. Other representative PDE4 or mixed PDE4/PDE3 inhibitors include AWD-12-281 (elbion); NCS-613 (INSERM); D-4418 (Chiroscience and Schering-Plough); CI-1018 or PD-168787 (Pfizer); benzodioxole compounds described in WO99/16766 (Kyowa Hakko); K-34 (Kyowa Hakko); V-11294A (Napp); roflumilast (Byk-Gulden); pthalazinone compounds described in WO99/47505 (Byk-Gulden); Pumafentrine (Byk-Gulden, now Altana); arofylline (Almirall-Prodesfarma); VM554/UM565 (Vemalis); T-440 (Tanabe Seiyaku); and T2585 (Tanabe Seiyaku).

Representative muscarinic antagonists (i.e., anticholinergic agents) that can be used in combination with the crystalline compounds of the invention include, but are not limited to, atropine, atropine sulfate, atropine oxide, methylatropine nitrate, homatropine hydrobromide, hyoscyamine (d, l) hydrobromide, scopolamine hydrobromide, ipratropium bromide, oxitropium bromide, tiotropium bromide, methantheline, propantheline bromide, anisotropine methyl bromide, clidinium bromide, copyrrolate (Robinul), isopropamide iodide, mepenzolate bromide, tridihexethyl chloride (Pathilone), hexocyclium methylsulfate, cyclopentolate hydrochloride, tropicamide, trihexyphenidyl hydrochloride, pirenzepine, telenzepine, AF-DX 116 and methoctramine and the like, or a pharmaceutically acceptable salt thereof; or, for those compounds listed as a salt, alternate pharmaceutically acceptable salt thereof.

Representative antihistamines (i.e., H₁-receptor antagonists) that can be used in combination with the crystalline compounds of the invention include, but are not limited to, ethanolamines such as carbinoxamine maleate, clemastine fumarate, diphenylhydramine hydrochloride and dimenhydrinate; ethylenediamines such as pyrilamine amleate, tripelennamine hydrochloride and tripelennamine citrate; alkylamines such as chlorpheniramine and acrivastine; piperazines such as hydroxyzine hydrochloride, hydroxyzine pamoate, cyclizine hydrochloride, cyclizine lactate, meclizine hydrochloride and cetirizine hydrochloride; piperidines such as astemizole, levocabastine hydrochloride, loratadine or its descarboethoxy analogue, terfenadine and fexofenadine hydrochloride; azelastine hydrochloride; and the like, or a pharmaceutically acceptable salt thereof; or, for those compounds listed as a salt, alternate pharmaceutically acceptable salt thereof.

Unless otherwise indicated, exemplary suitable doses for the other therapeutic agents administered in combination with a crystalline compound of the invention are in the range of about 0.05 μg/day to 100 mg/day.

The following formulations illustrate representative pharmaceutical compositions of the invention, as well as exemplary methods of preparation. One or more secondary agents can optionally be formulated with the crystalline compound of the invention (primary active agent). Alternately, the secondary agents(s) can be formulated separately and co-administered with the primary active agent, either simultaneously or sequentially. For example, in one embodiment, a single dry powder formulation can be manufactured to include both the crystalline compound of the invention and one or more secondary agents. In another embodiment, one formulation is manufactured to contain the crystalline compound of the invention and separate formulation(s) are manufactured to contain the secondary agent(s). Such dry powder formulations can then be packaged in separate blister packs and administered with a single DPI device.

### Exemplary Dry Powder Formulation for Administration by Inhalation

0.2 mg of a crystalline compound of the invention is micronized and then blended with 25 mg of lactose. The blended mixture is then loaded into a gelatin inhalation cartridge. The contents of the cartridge are administered using a powder inhaler.

### Exemplary Dry Powder Formulation for Administration by a Dry Powder Inhaler

A dry powder is prepared having a bulk formulation ratio of micronized crystalline compound of the invention (active agent) to lactose of 1:200. The powder is packed into a dry powder inhalation device capable of delivering between about 10 μg and 100 μg of active agent per dose.

### Exemplary Formulations for Administration by a Metered Dose Inhaler

A suspension containing 5 wt % of a crystalline compound of the invention (active agent) and 0.1 wt % lecithin is prepared by dispersing 10 g of the active agent as micronized particles with a mean size less than 10 μm in a solution formed from 0.2 g of lecithin dissolved in 200 mL of demineralized water. The suspension is spray dried and the resulting material is micronized to particles having a mean diameter less than 1.5 μm. The particles are loaded into cartridges with pressurized 1,1,1,2-tetrafluoroethane.

Alternately, a suspension containing 5 wt % of the active agent, 0.5 wt % lecithin, and 0.5 wt % trehalose is prepared by dispersing 5 g of the active agent as micronized particles with a mean size less than 10 μm in a colloidal solution formed from 0.5 g of trehalose and 0.5 g of lecithin dissolved in 100 mL of demineralized water. The suspension is spray dried and the resulting material is micronized to particles having a mean diameter less than 1.5 μm. The particles are loaded into canisters with pressurized 1,1,1,2-tetrafluoroethane.

### Exemplary Aqueous Aerosol Formulation for Administration by Nebulizer

A pharmaceutical composition is prepared by dissolving 0.5 mg of a crystalline compound of the invention (active agent) in 1 mL of a 0.9% sodium chloride solution acidified with citric acid. The mixture is stirred and sonicated until the active agent is dissolved. The pH of the solution is adjusted to a value in the range of from 3 to 8 (typically about 5) by the slow addition of NaOH.

### Exemplary Hard Gelatin Capsule Formulation for Oral Administration

The following ingredients are thoroughly blended and then loaded into a hard gelatin capsule: 250 mg of a crystalline compound of the invention, 200 mg of lactose (spray-dried), and 10 mg of magnesium stearate, for a total of 460 mg of composition per capsule.

### Exemplary Suspension Formulation for Oral Administration

The following ingredients are mixed to form a suspension containing 100 mg of active ingredient per 10 mL of suspension.

| Ingredients | Amount | |
|---|---|---|
| Crystalline compound of the invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum k (Vanderbilt Co.) | 1.0 | g |
| Flavoring | 0.035 | mL |
| Colorings | 0.5 | mg |
| Distilled water | q.s. to 100 | mL |

### Exemplary Injectable Formulation

The following ingredients are blended and the pH is adjusted to 4±0.5 using 0.5 N HCl or 0.5 N NaOH.

| Ingredients | Amount |
|---|---|
| Crystalline compound of the invention | 0.2 g |
| Sodium acetate buffer solution (0.4 M) | 2.0 mL |
| HCl (0.5 N) or NaOH (0.5 N) | q.s. to pH 4 |
| Water (distilled, sterile) | q.s. to 20 mL |

### Utility

The compound of formula I possesses muscarinic receptor antagonist activity and therefore, the crystalline naphthalene-1,5-disulfonic acid salts of the compound of formula I or solvates thereof, are expected to be useful for treating medical conditions mediated by muscarinic receptors, i.e., medical conditions which are ameliorated by treatment with a muscarinic receptor antagonist. Such medical conditions include, by way of example, pulmonary disorders or diseases including those associated with reversible airway obstruction such as chronic obstructive pulmonary disease (e.g., chronic, and wheezy bronchitis and emphysema), asthma, pulmonary fibrosis, allergic rhinitis, rhinorrhea, and the like. Other medical conditions that can be treated with muscarinic receptor antagonists are genitourinary tract disorders such as overactive bladder or detrusor hyperactivity and their symptoms; gastrointestinal tract disorders such as irritable bowel syndrome, diverticular disease, achalasia, gastrointestinal hypermotility disorders and diarrhea; cardiac arrhythmias such as sinus bradycardia; Parkinson's disease; cognitive disorders such as Alzheimer's disease; dismenorrhea; and the like.

Accordingly, in one embodiment, the invention is directed to a method for treating a pulmonary disorder, comprising administering to a patient a therapeutically effective amount of a crystalline naphthalene-1,5-disulfonic acid salt of biphenyl-2-ylcarbamic acid 1-{2-[5-(4-hydroxybenzylamino)pentylcarbamoyl]ethyl}piperidin-4-yl ester or solvate thereof. When used to treat a pulmonary disorder, the crystalline compound of the invention will typically be administered by inhalation in multiple doses per day, in a single daily dose or a single weekly dose. Generally, the dose for treating a pulmonary disorder will range from about 10 μg/day to 200 μg/day.

When administered by inhalation, the crystalline compounds of the invention typically have the effect of producing bronchodilation. Accordingly, in another embodiment, the invention is directed to a method of producing bronchodilation in a patient, comprising administering to a patient a bronchodilation-producing amount of a crystalline naphthalene-1,5-disulfonic acid salt of biphenyl-2-ylcarbamic acid 1-{2-[5-(4-hydroxybenzylamino)pentylcarbamoyl]ethyl}piperidin-4-yl ester or solvate thereof. Generally, the therapeutically effective dose for producing bronchodilation will range from about 10 μg/day to 200 μg/day.

In one embodiment, the invention is directed to a method of treating chronic obstructive pulmonary disease or asthma, comprising administering to a patient a therapeutically effective amount of a crystalline naphthalene-1,5-disulfonic acid salt of biphenyl-2-ylcarbamic acid 1-{2-[5-(4-hydroxybenzylamino)pentylcarbamoyl]ethyl}piperidin-4-yl ester or solvate thereof. When used to treat a COPD or asthma, the crystalline compound of the invention will typically be administered by inhalation in multiple doses per day or in a single daily dose. Generally, the dose for treating COPD or asthma will range from about 10 μg/day to 200 μg/day. As used herein, COPD includes chronic obstructive bronchitis and emphysema (see, for example, Barnes, Chronic Obstructive Pulmonary Disease (2000) N Engl J Med 343:269-78).

When used to treat a pulmonary disorder, the crystalline compounds of the invention are optionally administered in combination with other therapeutic agents. Accordingly, in a particular embodiment, the pharmaceutical compositions and methods of the invention further comprise a therapeutically effective amount of a β₂-adrenoreceptor agonist, a corticosteroid, a non-steroidal anti-inflammatory agent, or combination thereof.

In another embodiment, the crystalline compounds of the invention are used to antagonize a muscarinic receptor in a biological system, and a mammal in particular such as mice, rats, guinea pigs, rabbits, dogs, pigs, humans and so forth. In this embodiment, a therapeutically effective amount of a crystalline naphthalene-1,5-disulfonic acid salt of biphenyl-2-ylcarbamic acid 1-{2-[5-(4-hydroxybenzylamino)pentylcarbamoyl]ethyl}piperidin-4-yl ester or solvate thereof, is administered to the mammal. If desired, the effects of antagonizing the muscarinic receptor can then determined using conventional procedures and equipment.

The properties and utility of the crystalline compounds of the invention can be demonstrated using various in vitro and in vivo assays that are well-known to those skilled in the art. For example, representative assays are described in further detail in the following Examples.

### EXAMPLES

The following Preparations and Examples are provided to illustrate specific embodiments of the invention. These specific embodiments, however, are not intended to limit the scope of the invention in any way unless specifically indicated.

The following abbreviations have the following meanings unless otherwise indicated and any other abbreviations used herein and not defined have their standard meaning:
AC adenylyl cyclase
ACh acetylcholine
ACN acetonitrile
BSA bovine serum albumin
cAMP 3′-5′ cyclic adenosine monophosphate
CHO Chinese hamster ovary
cM5 cloned chimpanzee M5 receptor
DCM dichloromethane (i.e., methylene chloride)
DIPEA N,N-diisopropylethylamine
dPBS Dulbecco's phosphate buffered saline
EDTA ethylenediaminetetraacetic acid
EtOAc ethyl acetate
EtOH ethanol
FBS fetal bovine serum
FLIPR fluorometric imaging plate reader
HATU O-(7 azabenzotriazol-1-yl-N,N,N′,N′-tetramethyluronium hexafluorophosphate
HBSS Hank's buffered salt solution
HEPES 4-(2-hydroxyethyl)-1-piperazineethanesulfonic acid
hM₁ cloned human M1 receptor
hM₂ cloned human M2 receptor
hM₃ cloned human M3 receptor
hM₄ cloned human M4 receptor
hM₅ cloned human M5 receptor
HPLC high-performance liquid chromatography
MCh methylcholine
MeOH methanol
TFA trifluoroacetic acid
THF tetrahydrofuran

Any other abbreviations used herein but not defined have their standard, generally accepted meaning. Unless noted otherwise, reagents, starting materials and solvents were purchased from commercial suppliers (such as Sigma-Aldrich, Fluka, and the like) and were used without further purification.

In the examples described below, HPLC analysis was conducted using an Agilent (Palo Alto, Calif.) Series 1100 instrument with Zorbax Bonus RP 2.1×50 mm columns, supplied by Agilent, (a C14 column), having a 3.5 micron particle size. Detection was by UV absorbance at 214 nm. HPLC 10-70 data was obtained with a flow rate of 0.5 mL/minute of 10%-70% B over 6 minutes. Mobile phase A was 2%-98%-0.1% ACN—H₂O-TFA; and mobile phase B was 90%-10%-0.1% ACN—H₂O-TFA. Using the mobile phases A and B described above, HPLC₅-35 data and HPLC 10-90 data were obtained with a 5 minute gradient.

Liquid chromatography mass spectrometry (LCMS) data were obtained with an Applied Biosystems (Foster City, Calif.) model API-150EX instrument. LCMS 10-90 data was obtained with a 10%-90% mobile phase B over a 5 minute gradient.

Small scale purification was conducted using an API-150EX Prep Workstation system from Applied Biosystems. The mobile phase was A: water+0.05% v/v TFA; and B: acetonitrile+0.05% v/v TFA. For arrays (typically about 3 to 50 mg recovered sample size) the following conditions were used: 20 mL/min flow rate; 15 minute gradients and a 20 mm×50 mm Prism RP column with 5 micron particles (Thermo Hypersil-Keystone, Bellefonte, Pa.). For larger scale purifications (typically greater than 100 mg crude sample), the following conditions were used: 60 mL/min flow rate; 30 minute gradients and a 41.4 mm×250 mm Microsorb BDS column with 10 micron particles (Varian, Palo Alto, Calif.).

### Preparation 1

### Biphenyl-2-ylcarbamic Acid Piperidin-4-yl Ester

Biphenyl-2-isocyanate (97.5 g, 521 mmol) and 4-hydroxy-N-benzylpiperidine (105 g, 549 mmol) were heated together at 70° C. for 12 hours. The reaction mixture was then cooled to 50° C. and EtOH (1 L) was added and then 6M HCl (191 mL) was added slowly. The resulting mixture was then cooled to ambient temperature and ammonium formate (98.5 g, 1.56 mol) was added and then nitrogen gas was bubbled through the solution vigorously for 20 minutes. Palladium on activated carbon (20 g, 10 wt % dry basis) was then added and the reaction mixture was heated at 40° C. for 12 hours, and then filtered through a pad of Celite. The solvent was then removed under reduced pressure and 1M HCl (40 mL) was added to the crude residue. The pH of the mixture was then adjusted with 10 N NaOH to pH 12. The aqueous layer was extracted with EtOAc (2×150 mL) and the organic layer was dried (magnesium sulfate), filtered and the solvent removed under reduced pressure to give 155 g of the title intermediate (100% yield). HPLC (10-70) Rₜ=2.52; m/z: [M+H⁺] calc'd for C₁₈H₂₀N₂O₂ 297.15; found 297.3.

### Preparation 2

### 3-[4-(Biphenyl-2-ylcarbamoyloxy)piperidin-1-yl]propionic Acid

A round bottomed flask was charged with biphenyl-2-ylcarbamic acid piperidin-4-yl ester (50 g, 67.6 mmol, 1 equiv.; prepared as described in Preparation 1) and 500 mL of DCM. Acrylic acid (15.05 mL, 100 mmol, 1.3 equiv.) was added and the reaction was heated to 50° C. (reflux) for 18 hours. The DCM was removed in vacuo and MeOH (600 mL) was added. The MeOH solution was heated to 75° C. for 2 hours then allowed to cool to room temperature, during which time a thick slurry formed. The precipitate was collected via vacuum filtration, washed with MeOH (50 mL), and dried on the filter to afford 61 g of title intermediate (98% yield).

### Preparation 3

### Biphenyl-2-ylcarbamic Acid 1-[2-(5-Aminopentylcarbamoyl)ethyl]piperidin-4-yl Ester

A mixture of 3-[4-(biphenyl-2-ylcarbamoyloxy)piperidin-1-yl]propionic acid (98.6 g, 267 mmol, 1.0 equiv.; prepared as described in Preparation 2), diphenylphosphoryl azide (69 mL, 321 mmol, 1.2 equiv.), N-tert-butoxycarbonyl-1,5-diaminopentane (65.0 g, 321 mmol, 1.2 equiv.) and DIPEA (93 mL, 534 mmol, 2.0 equiv.) in DCM (790 mL) was stirred at room temperature overnight. When the coupling was complete (determined by HPLC), 4.0 M HCl in dioxane was added (267 mL, 1.068 mol, 4.0 equiv.) and the reaction was stirred overnight. The reaction was diluted with water (1.0 L) and transferred to a separatory funnel. The DCM layer was removed and the aqueous layer was washed with isopropyl acetate (200 mL). The pH of the aqueous layer was adjusted to 13-14 with solid NaOH, and the basic aqueous layer was extracted 3× isopropyl acetate (250 mL). Combined organic layers with washed with saturated brine (500 mL) and dried over anhydrous sodium sulfate. Solvent was removed to afford 126 g of crude title compound that was immediately carried forward to Example 1.

### Example 1

### Biphenyl-2-ylcarbamic Acid 1-{2-[5-(4-Hydroxybenzylamino)pentylcarbamoyl]ethyl}piperidin-4-yl Ester

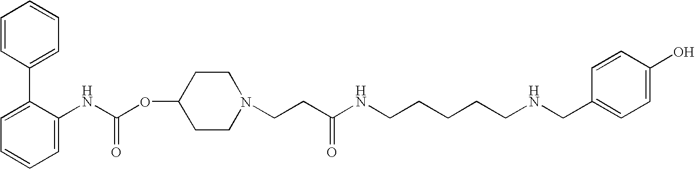

To a 0.1 M solution of biphenyl-2-ylcarbamic acid 1-[2-(5-aminopentylcarbamoyl) ethyl]piperidin-4-yl ester (126 g, 287 mmol, 1.0 equiv.; prepared as described in Preparation 3) in anhydrous MeOH (2.87 L) was added 4-hydroxybenzaldehyde (44.2 g, 362 mmol, 1.3 equiv.) and the resulting mixture was stirred for 1 hour. Sodium triacetoxyborohydride (118 g, 557 mmol, 2.0 equiv.) was then added and the reaction mixture was stirred at room temperature for 1 hour. Additional sodium triacetoxyborohydride (59 g, 278 mmol, 1.0 equiv.) was added and the reaction was stirred for an additional 2 hours. The reaction was monitored by HPLC. When complete, the reaction was diluted with 1N aqueous HCl (6.0 L) and isopropyl acetate (3 L). The reaction was mixed and the layers were separated. The aqueous layer was washed again with isopropyl acetate (1 L). The pH of the aqueous layer was then adjusted to 13-14 by addition of solid NaOH, and the basic aqueous layer was extracted 3× isopropyl acetate (1 L). The extraction layers were combined, washed with saturated brine, and dried over anhydrous sodium sulfate. Drying agent was removed by filtration and solvent was removed in vacuo to afford 136 g of crude title compound. MS m/z: [M+H⁺] calc'd for C₃₃H₄₂N₄O₄ 559.3; found 559.6.

### Example 2

### Crystalline Mononapadisylate Salt of Biphenyl-2-ylcarbamic Acid 1-2-[5-(4-Hydroxybenzylamino)pentylcarbamoyl]ethyl]piperidin-4-yl Ester Via Heminapadisylate Salt Intermediate

Reagents:

Crystallization Solvent: 75% ACN, 25% MeOH, v/v

Wash Solvent: 90% ACN, 10% MeOH, v/v

Free Acid Solution: 37.3 g of 1,5 naphthalene disulfonic acid anhydrous in 3.6 L of Crystallization Solvent, filtered (0.22 μm Millipore vacuum filter) and stored at 40° C. for later use.

Crystallizer: A 15 liter cylindrical Pyrex glass vessel was used as a crystallizer. This had a bottom draw off for slurry collection, with minimal dead volume. Agitation was achieved with an overhead agitator drive and a 45 degree fixed pitch, four bladed impeller. The impeller was situated close to the base of the vessel and was rotated with an overhead drive in an upflow direction.

Filtration: A 10 L vacuum flask with a 200 mm Buchner funnel filter (Whatman media) was used for slurry filtration and crystal washing.

Procedure: Reactions were all conducted at room temperature (22° C.). Crude free base (55 g crude free base and 48 g pure free base, 87.6% pure), prepared as described in Example 1, was dissolved in 700 mL of Crystallization Solvent (700 mL), filtered (0.22 μm Millipore vacuum filter) and diluted with a further 1.7 L of Crystallization Solvent. This free base solution (2.4 L) and 1.2 L of Free Acid Solution were simultaneously charged to the crystallizer over 5 minutes, whilst agitating at 168 rpm. Agitation was then reduced to 150 rpm. Nucleation of the crystalline heminapadisylate occurred within 15 minutes. An additional 1.2 L of Free Acid Solution was charged over 5 minutes. Agitation of the slurry was continued at 150 rpm for an additional 120 minutes. The slurry was discharged from the crystallizer, filtered, and the filter cake was suction dried in place for 15 minutes. The crystals were separated from the filter media and weighed (41.7 g, 0.2 g of this was withheld for other analyses).

The empty crystallizer was cleaned using 300 mL of MeOH. The remaining 41.5 g of crystal product was resuspended in the crystallizer in 6 L of fresh Crystallization Solvent and agitated at 150 rpm. 1.2 L of the Free Acid Solution was charged to the crystallizer over 5 minutes and agitation continued overnight. The slurry was filtered 21 hours after resuspension, washed with 500 mL of Wash Solvent, then suction dried for 1 hour. The mass of the mononapadisylate crystal dry cake was 35.86 g; solids were transferred to a vial and nitrogen purged overnight.

### Example 3

### Crystalline Mononapadisylate Salt of Biphenyl-2-ylcarbamic Acid 1-[2-[5-(4-Hydroxybenzylamino)pentylcarbamoyl]ethyl]piperidin-4-yl Ester

To a scintillation vial, 249 mg of free base, prepared as described in Example 1, and 127 mg of anhydrous 1,5-naphthalene disulfonic acid was added. These solids were dissolved in 3.4 mL of MeOH at 65° C. ACN antisolvent (8.6 mL) was added, followed by cooling of the vial to room temperature and holding overnight. Several large crystals formed. The mother liquor was decanted into a separate vial. Further ACN antisolvent was added (2.0 mL), which resulted in clouding. The vial was cooled to 4° C. and a further 1 mL of ACN was added. After 2 hours, 173 mg of mononapadisylate crystals was recovered by filtration. The crystalline solids were dried for 10 minutes in an oven at 85° C.

### Example 4

### Crystalline Mononapadisylate Ethanolate of Biphenyl-2-ylcarbamic Acid 1-{2-[5-(4-Hydroxybenzylamino)pentylcarbamoyl]ethyl}piperidin-4-yl Ester

A 100 mL round bottom flask equipped with an overhead stirrer was charged with 1.0 g freebase, prepared as described in Example 1, and 0.485 g anhydrous 1,5-naphthalene disulfonic acid. The solids were heated to 60° C. in 16.75 mL methanol until solids had dissolved. The solution was agitated at 100 rpm. EtOH was added (8.25 mL) over 30 minutes. The temperature was decreased to 45° C. over 30 minutes, then to 40° C. for the following 30 minutes and finally to room temperature and held for 17 hours. A dense thick white slurry of mononapadisylate ethanolate crystals existed at this time point. A small quantity of solids were isolated for analysis by withdrawing 2 mL of this slurry and filtering off the mother liquor.

### Example 5

### Powder X-Ray Diffraction

Powder X-ray diffraction patterns were obtained with a Rigaku diffractometer using Cu Kα (30.0 kV, 15.0 mA) radiation. The analysis was performed with the goniometer running in continuous-scan mode of 3° per minute with a step size of 0.03° over a range of 2 to 45°. Samples were prepared on quartz specimen holders as a thin layer of powdered material. The instrument was calibrated with a silicon metal standard.

The PXRD patterns for the compounds synthesized herein showed the materials to be crystalline. A representative PXRD pattern for a sample of the mononapadisylate salt of Example 2 is shown in FIG. 1. A representative PXRD pattern for a sample of the crystalline heminapadisylate salt of Example 2 is shown in FIG. 6. A representative PXRD pattern for a sample of the crystalline mononapadisylate ethanolate of Example 4 is shown in FIG. 11.

### Example 6

### Thermal Analysis

Differential scanning calorimetry (DSC) was performed using a TA Instruments Model Q-10 module with a Thermal Analyst controller. Data were collected and analyzed using TA Instruments Thermal Solutions software. A sample of about 1 mg was accurately weighed into an aluminum pan with lid. The sample was evaluated using a linear heating ramp of 10° C./min from ambient temperature to approximately 250° C. The DSC cell was purged with dry nitrogen during use.

A representative DSC trace for a sample of the crystalline mononapadisylate salt of Example 2 showed a transition at about 166° C., as seen in FIG. 2. This DSC trace demonstrate that this crystalline salt has excellent thermal stability with the melting peak at about 176° C. and no thermal decomposition below 225° C. The DSC trace also showed an onset of endothermic heat flow at about 165° C.

A representative DSC trace for a sample of the crystalline heminapadisylate salt of Example 2 showed a transition at about 160° C., as seen in FIG. 7. This DSC trace demonstrate that this crystalline salt has excellent thermal stability with the melting peak at about 160° C. and no thermal decomposition below 225° C. The DSC trace also showed an onset of endothermic heat flow at about 145° C.

A representative DSC trace for a sample of the crystalline mononapadisylate ethanolate of Example 4 showed a transition at about 167° C., as seen in FIG. 12. The DSC trace demonstrated that this crystalline salt has excellent thermal stability with the melting peak at about 167° C.

Thermogravimetric analysis (TGA) was performed using a TA Instruments Model Q-50 module equipped with high resolution capability. Data were collected and analyzed using TA Instruments Thermal Solutions software. A sample weighing about 2 mg was placed onto a platinum pan and scanned with a high resolution-heating rate from ambient temperature to 300° C. The balance and furnace chambers were purged with nitrogen flows during use. A representative TGA trace for a sample of the crystalline mononapadisylate salt of Example 2 showed a loss of solvents and/or water (4.5%) at temperatures below 70° C., as seen in FIG. 3. This TGA trace indicate that the crystalline compounds of the present invention lose a small amount of weight from room temperature to moderately elevated temperatures, which is consistent with the loss of residual moisture or solvent.

### Example 7

### Dynamic Moisture Sorption Assessment

A dynamic moisture sorption (DMS) assessment (also known as a moisture sorption-desorption profile) was performed for samples of the crystalline naphthalene-1,5-disulfonic acid salts using a VTI atmospheric microbalance, SGA-100 system (VTI Corp., Hialeah, Fla. 33016). A sample size of approximately 10 mg was used and the humidity was set at the ambient value at the start of the analysis. A typical DMS analysis consisted of three scans: ambient to 2% relative humidity (RH), 2% RH to 90% RH, 90% RH to 5% RH at a scan rate of 5% RH/step. The mass was measured every two minutes and the RH was changed to the next value (+/−5% RH) when the mass of the sample was stable to within 0.01% for 5 consecutive points.

A representative DMS trace for a sample of the crystalline mononapadisylate salt of Example 2 showed a reversible sorption/desorption profile with low hygroscopicity, with a 4.5% weight gain when exposed to 2-90% RH and a 0.8% weight gain in the humidity range of 40-75% RH, as shown in FIG. 4. A representative DMS trace for a sample of the crystalline heminapadisylate salt of Example 2 showed a reversible sorption/desorption profile with low hygroscopicity, with a 4.5% weight gain when exposed to 2-90% RH and a 2.1% weight gain in the humidity range of 40-75% RH, as shown in FIG. 9.

These DMS traces demonstrated that the crystalline compounds of the present invention have a reversible sorption/desorption profile with low hygroscopicity. The crystalline compounds have an acceptable weight gain when exposed to a broad humidity range. The reversible moisture sorption/desorption profiles demonstrate that the crystalline compounds of the present invention possess an acceptable hygroscopicity and are not deliquescent.

### Example 8

### Elemental Analysis

The following elemental percentages for a sample of the crystalline mononapadisylate salt of Example 2 were determined by combustion analysis using a Flash EA 1112 Elemental Analyzer (CE Elantech, Lakewood, N.J.): carbon 57.58%, hydrogen 6.21%, nitrogen 6.09%, and sulfur 6.98% (results); carbon 60.97%, hydrogen 5.95%, nitrogen 6.61%, and sulfur 7.57% (expected). The difference is accounted for by the residual moisture content (5.13%)

### Example 9

### Solid State Stability Assessment

Samples of the crystalline mononapadisylate salt of Example 2, about 200 mg each, were stored in multiple 3 mL borosilicate vials at −20° C. (closed container), 40° C./75% RH (open and closed container), and at 50° C. (closed container). At specific intervals, the entire contents of a representative vial was analyzed by the following HPLC method:

Column: Agilent Zorbox SB-C18, 4.6×250 mm, 5 μm; Mobile Phase A: 98% water, 2% ACN, 0.1% TFA; Mobile Phase B: 10% water, 90% ACN; 0.1% TFA; Flow rate: 1 mL/min; Injection Volume: 20 μL; Detector: 220 nm; Gradient-Time in minutes (% Mobile Phase B): 0.0 (10); 4.00 (20); 26.00 (28); 34.40 (100); 38.40 (100); 38.50 (10); and 45.00 (10). Samples were prepared as 10 mg/mL stock solutions in 10-50% ACN in H₂O, depending on the solubility. These stock solutions were diluted to 1 mg/mL in 10% ACN for injection onto the HPLC.

The initial purity of the samples was 99.8% as determined by HPLC area percentage. After 6 weeks of storage, for the samples kept under all conditions, there was no detectable change in chemical purity, no observable change in the appearance of the material, and analysis by DSC and TGA showed no detectable differences.

### Example 10

### Micronization

A 10 g sample of the crystalline mononapadisylate salt of Example 2 was micronized to give 6.4 g of a free-flowing white powder (64% recovery). Pre-micronization, the crystalline mononapadisylate had an initial purity of 99.8% as determined by HPLC area percentage. The purity of the micronized material was the same. The water content of the pre-micronized material was 5.13 wt %, and the water content of the micronized material was 3.75 wt %. The particle size distribution was as follows:

| | Pre-Micronization | Post-Micronization |
|---|---|---|
| D (v, 0.9) | 22.1 μm | 3.9 μm |
| D (v, 0.5) | 9.4 μm | 1.6 μm |
| D (v, 0.1) | 2.5 μm | 0.4 μm |

No significant changes were observed in the powder x-ray diffraction pattern, TGA, DSC, DMS, chemical purity, chiral purity and moisture content for the micronized material compared to the unmicronized material. For example, a sample of the crystalline mononapadisylate salt of Example 2 showed a 0.7% weight gain in the humidity range of 40-70% RH, while the micronized material showed a 1.0% weight gain in this range.

### Example 11

### Inhalation Solution Stability

A solution was prepared with 0.1 mg/mL freebase equivalents (using a crystalline mononapadisylate salt prepared as described in Example 2) in 10 mM citrate buffered normal saline, pH 4.5. The solubility of the crystalline salt was approximately 0.4 mg/mL in the buffer. Less than 0.5% degradation was observed after storage for one month at 40° C./75% RH.

### ASSAY 1

### Radioligand Binding Assay

Membrane Preparation from Cells Expressing hM₁, hM₂, hM₃ and hM₄ Muscarinic Receptor Subtypes

CHO cell lines stably expressing cloned human hM₁, hM₂, hM₃ and hM₄ muscarinic receptor subtypes, respectively, were grown to near confluency in medium consisting of HAM's F-12 supplemented with 10% FBS and 250 μg/mL Geneticin. The cells were grown in a 5% CO₂, 37° C. incubator and lifted with 2 mM EDTA in DPBS. Cells were collected by 5 minute centrifugation at 650×g, and cell pellets were either stored frozen at −80° C. or membranes were prepared immediately. For membrane preparation, cell pellets were resuspended in lysis buffer and homogenized with a Polytron PT-2100 tissue disrupter (Kinematica AG; 20 seconds×2 bursts). Crude membranes were centrifuged at 40,000×g for 15 minutes at 4° C. The membrane pellet was then resuspended with resuspension buffer and homogenized again with the Polytron tissue disrupter. The protein concentration of the membrane suspension was determined by the method described in Lowry, O. et al., Journal of Biochemistry 193:265 (1951). All membranes were stored frozen in aliquots at −80° C. or used immediately. Aliquots of prepared hM₅ receptor membranes were purchased directly from Perkin Elmer and stored at −80° C. until use.

### Radioligand Binding Assay on Muscarinic Receptor Subtypes hM₁, hM₂, hM₃, hM₄ and hM₅

Radioligand binding assays were performed in 96-well microtiter plates in a total assay volume of 100 μL. CHO cell membranes stably expressing either the hM₁, hM₂, hM₃, hM₄ or hM₅ muscarinic subtype were diluted in assay buffer to the following specific target protein concentrations (μg/well): 10 μg for hM₁, 10-15 μg for hM₂, 10-20 μg for hM₃, 10-20 μg for hM₄, and 10-12 μg for hM₅. The membranes were briefly homogenized using a Polytron tissue disruptor (10 seconds) prior to assay plate addition. Saturation binding studies for determining K_{D} values of the radioligand were performed using L-[N-methyl-³H]scopolamine methyl chloride ([³H]-NMS) (TRK666, 84.0 Ci/mmol, Amersham Pharmacia Biotech, Buckinghamshire, England) at concentrations ranging from 0.001 nM to 20 nM. Displacement assays for determination of Kᵢ values of test compounds were performed with [³H]-NMS at 1 nM and eleven different test compound concentrations. The test compounds were initially dissolved to a concentration of 400 μM in dilution buffer and then serially diluted 5× with dilution buffer to final concentrations ranging from 10 μM to 100 μM. The addition order and volumes to the assay plates were as follows: 25 μL radioligand, 25 μL diluted test compound, and 50 μL membranes. Assay plates were incubated for 60 minutes at 37° C. Binding reactions were terminated by rapid filtration over GF/B glass fiber filter plates (PerkinElmer Inc., Wellesley, Mass.) pre-treated in 1% BSA. Filter plates were rinsed three times with wash buffer (10 mM HEPES) to remove unbound radioactivity. Plates were then air dried, and 50 μL Microscint-20 liquid scintillation fluid (PerkinElmer Inc., Wellesley, Mass.) was added to each well. The plates were then counted in a PerkinElmer Topcount liquid scintillation counter (PerkinElmer Inc., Wellesley, Mass.). Binding data were analyzed by nonlinear regression analysis with the GraphPad Prism Software package (GraphPad Software, Inc., San Diego, Calif.) using the one-site competition model. Kᵢ values for test compounds were calculated from observed IC₅₀ values and the K_{D} value of the radioligand using the Cheng-Prusoff equation (Cheng Y; Prusoff W. H. Biochemical Pharmacology 22(23):3099-108 (1973)). Kᵢ values were converted to pKᵢ values to determine the geometric mean and 95% confidence intervals. These summary statistics were then converted back to Kᵢ values for data reporting.

In this assay, a lower Kᵢ value indicates that the test compound has a higher binding affinity for the receptor tested. The compound of formula I was found to have a Kᵢ value of less than about 5 nM for the M₃ muscarinic receptor subtype when tested in this or a similar assay.

### Assay 2

### Muscarinic Receptor Functional Potency Assays

### Blockade of Agonist-Mediated Inhibition of cAMP Accumulation

In this assay, the functional potency of a test compound was determined by measuring the ability of the test compound to block oxotremorine-inhibition of forskolin-mediated cAMP accumulation in CHO-K1 cells expressing the hM₂ receptor. cAMP assays were performed in a radioimmunoassay format using the Flashplate Adenylyl Cyclase Activation Assay System with ¹²⁵I-cAMP (NEN SMP004B, PerkinElmer Life Sciences Inc., Boston, Mass.), according to the manufacturer's instructions.

Cells were rinsed once with dPBS and lifted with Trypsin-EDTA solution (0.05% trypsin/0.53 mM EDTA) as described in the Cell Culture and Membrane Preparation section above. The detached cells were washed twice by centrifugation at 650×g for five minutes in 50 mLs dPBS. The cell pellet was then re-suspended in 10 mL dPBS, and the cells were counted with a Coulter ZI Dual Particle Counter (Beckman Coulter, Fullerton, Calif.). The cells were centrifuged again at 650×g for five minutes and re-suspended in stimulation buffer to an assay concentration of 1.6×10⁶-2.8×10⁶ cells/mL.

The test compound was initially dissolved to a concentration of 400 μM in dilution buffer (+supplemented with 1 mg/mL BSA (0.1%)), and then serially diluted with dilution buffer to final molar concentrations ranging from 100 μM to 0.1 nM. Oxotremorine was diluted in a similar manner.

To measure oxotremorine inhibition of AC activity, 25 μL forskolin (25 μM final concentration diluted in dPBS), 25 μL diluted oxotremorine, and 50 μL cells were added to agonist assay wells. To measure the ability of a test compound to block oxotremorine-inhibited AC activity, 25 μL forskolin and oxotremorine (25 μM and 5 μM final concentrations, respectively, diluted in dPBS) 25 μL diluted test compound, and 50 μL cells were added to remaining assay wells. Reactions were incubated for 10 minutes at 37° C. and stopped by addition of 100 μL ice-cold detection buffer. Plates were sealed, incubated overnight at room temperature and counted the next morning on a PerkinElmer TopCount liquid scintillation counter (PerkinElmer Inc., Wellesley, Mass.). The amount of cAMP produced (pmol/well) was calculated based on the counts observed for the samples and cAMP standards, as described in the manufacturer's user manual. Data were analyzed by nonlinear regression analysis with the GraphPad Prism Software package (GraphPad Software, Inc., San Diego, Calif.) using the non-linear regression, one-site competition equation. The Cheng-Prusoff equation was used to calculate the Kᵢ, using the EC₅₀ of the oxotremorine concentration-response curve and the oxotremorine assay concentration as the K_{D} and [L], respectively. The Kᵢ values were converted to pKᵢ values to determine the geometric mean and 95% confidence intervals. These summary statistics were then converted back to Kᵢ values for data reporting.

In this assay, a lower Kᵢ value indicates that the test compound has a higher functional activity at the receptor tested. The compound of formula I was found to have a Kᵢ value of less than about 10 nM for blockade of oxotremorine-inhibition of forskolin-mediated cAMP accumulation in CHO-K1 cells expressing the hM₂ receptor, when tested in this or a similar assay.

### Blockade of Agonist-Mediated [³⁵S]GTPγS Binding

In a second functional assay, the functional potency of test compounds can be determined by measuring the ability of the compounds to block oxotremorine-stimulated [³⁵S]GTPγS binding in CHO-K1 cells expressing the hM₂ receptor.

At the time of use, frozen membranes were thawed and then diluted in assay buffer with a final target tissue concentration of 5-10 μg protein per well. The membranes were briefly homogenized using a Polytron PT-2100 tissue disrupter and then added to the assay plates. The EC₉₀ value (effective concentration for 90% maximal response) for stimulation of [³⁵S]GTPγS binding by the agonist oxotremorine was determined in each experiment.

To determine the ability of a test compound to inhibit oxotremorine-stimulated [³⁵S]GTPγS binding, the following was added to each well of 96 well plates: 25 μL of assay buffer with [³⁵S]GTPγS (0.4 nM), 25 μL of oxotremorine (EC₉₀) and GDP (3 μM), 25 μL of diluted test compound and 25 μL CHO cell membranes expressing the hM₂ receptor. The assay plates were then incubated at 37° C. for 60 minutes. The assay plates were filtered over 1% BSA-pretreated GF/B filters using a PerkinElmer 96-well harvester. The plates were rinsed with ice-cold wash buffer for 3×3 seconds and then air or vacuum dried. Microscint-20 scintillation liquid (50 μL) was added to each well, and each plate was sealed and radioactivity counted on a topcounter (PerkinElmer). Data were analyzed by nonlinear regression analysis with the GraphPad Prism Software package (GraphPad Software, Inc., San Diego, Calif.) using the non-linear regression, one-site competition equation. The Cheng-Prusoff equation was used to calculate the Kᵢ, using the IC₅₀ values of the concentration-response curve for the test compound and the oxotremorine concentration in the assay as the K_{D} and [L], ligand concentration, respectively.

In this assay, a lower Kᵢ value indicates that the test compound has a higher functional activity at the receptor tested. The compound of formula I was found to have a Kᵢ value of less than about 10 nM for blockade of oxotremorine-stimulated [³⁵S]GTPγS binding in CHO-K1 cells expressing the hM₂ receptor, when tested in this or a similar assay.

### Blockade of Agonist-Mediated Calcium Release via FLIPR Assays

Muscarinic receptor subtypes (M₁, M₃ and M₅ receptors), which couple to G_{q} proteins, activate the phospholipase C (PLC) pathway upon agonist binding to the receptor. As a result, activated PLC hydrolyzes phosphatyl inositol diphosphate (PIP₂) to diacylglycerol (DAG) and phosphatidyl-1,4,5-triphosphate (IP₃), which in turn generates calcium release from intracellular stores, i.e., endoplasmic and sarcoplasmic reticulum. The FLIPR (Molecular Devices, Sunnyvale, Calif.) assay capitalizes on this increase in intracellular calcium by using a calcium sensitive dye (Fluo-4AM, Molecular Probes, Eugene, Oreg.) that fluoresces when free calcium binds. This fluorescence event was measured in real time by the FLIPR, which detected the change in fluorescence from a monolayer of cells cloned with human M₁ and M₃, and chimpanzee M₅ receptors. Antagonist potency was determined by the ability of antagonists to inhibit agonist-mediated increases in intracellular calcium.

For FLIPR calcium stimulation assays, CHO cells stably expressing the hM₁, hM₃ and cM₅ receptors were seeded into 96-well FLIPR plates the night before the assay was done. Seeded cells were washed twice by Cellwash (MTX Labsystems, Inc.) with FLIPR buffer (10 mM HEPES, pH 7.4, 2 mM calcium chloride, 2.5 mM probenecid in HBSS without calcium and magnesium) to remove growth media and leaving 50 μL/well of FLIPR buffer. The cells were then incubated with 50 μL/well of 4 μM FLUO-4AM (a 2× solution was made) for 40 minutes at 37° C., 5% carbon dioxide. Following the dye incubation period, cells were washed two times with FLIPR buffer, leaving a final volume of 50 μL/well.

To determine antagonist potency, the dose-dependent stimulation of intracellular Ca²⁺ release for oxotremorine was first determined so that antagonist potency can later be measured against oxotremorine stimulation at an EC₉₀ concentration. Cells were first incubated with compound dilution buffer for 20 minutes, followed by agonist addition, which is performed by the FLIPR. An EC₉₀ value for oxotremorine was generated according to the method detailed in the FLIPR measurement and data reduction section below, in conjunction with the formula EC_{F}=((F/100−F)ˆ1/H)*EC₅₀. An oxotremorine concentration of 3×ECF is prepared in stimulation plates such that an EC₉₀ concentration of oxotremorine was added to each well in the antagonist inhibition assay plates.

The parameters used for the FLIPR were: exposure length of 0.4 seconds, laser strength of 0.5 watts, excitation wavelength of 488 nm, and emission wavelength of 550 nm. Baseline was determined by measuring the change in fluorescence for 10 seconds prior to addition of agonist. Following agonist stimulation, the FLIPR continuously measured the change of fluorescence every 0.5 to 1 second for 1.5 minutes to capture the maximum fluorescence change. The change of fluorescence was expressed as maximum fluorescence minus baseline fluorescence for each well. The raw data was analyzed against the logarithm of drug concentration by nonlinear regression with GraphPad Prism (GraphPad Software, Inc., San Diego, Calif.) using the built-in model for sigmoidal dose-response. Antagonist Kᵢ values were determined by Prism using the oxotremorine EC₅₀ value as the K_{D} and the oxotremorine EC₉₀ for the ligand concentration according to the Cheng-Prusoff equation (Cheng & Prusoff, 1973).

In this assay, a lower Kᵢ value indicates that the test compound has a higher functional activity at the receptor tested. The compound of formula I was found to have a Kᵢ value of less than about 10 nM for blockade of agonist-mediated calcium release in CHO cells stably expressing the hM₃ receptor, when tested in this or a similar assay.

### Assay 3

### Determination of Duration of Bronchoprotection in Guinea Pig Model of Acetylcholine-Induced Bronchoconstriction

This in vivo assay is used to assess the bronchoprotective effects of test compounds exhibiting muscarinic receptor antagonist activity. Groups of six male guinea pigs (Duncan-Hartley (HsdPoc:DH) Harlan, Madison, Wis.) weighing between 250 and 350 g are individually identified by cage cards. Throughout the study animals are allowed access to food and water ad libitum.

Test compounds are administered via inhalation over 10 minutes in a whole-body exposure dosing chamber (R&S Molds, San Carlos, Calif.). The dosing chambers are arranged so that an aerosol was simultaneously delivered to 6 individual chambers from a central manifold. Guinea pigs are exposed to an aerosol of a test compound or vehicle (WFI). These aerosols are generated from aqueous solutions using an LC Star Nebulizer Set (Model 22F51, PAR1 Respiratory Equipment, Inc. Midlothian, Va.) driven by a mixture of gases (CO₂=5%, O₂=21% and N₂=74%) at a pressure of 22 psi. The gas flow through the nebulizer at this operating pressure is approximately 3 L/minute. The generated aerosols are driven into the chambers by positive pressure. No dilution air is used during the delivery of aerosolized solutions. During the 10 minute nebulization, approximately 1.8 mL of solution is nebulized. This is measured gravimetrically by comparing pre-and post-nebulization weights of the filled nebulizer.

The bronchoprotective effects of test compounds administered via inhalation are evaluated using whole body plethysmography at 1.5, 24, 48 and 72 hours post-dose.

Forty-five minutes prior to the start of the pulmonary evaluation, each guinea pig is anesthetized with an intramuscular injection of ketamine (43.75 mg/kg), xylazine (3.50 mg/kg) and acepromazine (1.05 mg/kg). After the surgical site is shaved and cleaned with 70% alcohol, a 2-3 cm midline incision of the ventral aspect of the neck was made. Then, the jugular vein is isolated and cannulated with a saline-filled polyethylene catheter (PE-50, Becton Dickinson, Sparks, Md.) to allow for intravenous infusions of ACh (Sigma-Aldrich, St. Louis, Mo.) in saline. The trachea is then dissected free and cannulated with a 14G teflon tube (#NE-014, Small Parts, Miami Lakes, Fla.). If required, anesthesia is maintained by additional intramuscular injections of the aforementioned anesthetic mixture. The depth of anesthesia is monitored and adjusted if the animal responds to pinching of its paw or if the respiration rate is greater than 100 breaths/minute.

Once the cannulations are complete, the animal is placed into a plethysmograph (#PLY3114, Buxco Electronics, Inc., Sharon, Conn.) and an esophageal pressure cannula (PE-160, Becton Dickinson, Sparks, Md.) is inserted to measure pulmonary driving pressure (pressure). The teflon tracheal tube is attached to the opening of the plethysmograph to allow the guinea pig to breathe room air from outside the chamber. The chamber is then sealed. A heating lamp is used to maintain body temperature and the guinea pig's lungs are inflated 3 times with 4 mL of air using a 10 mL calibration syringe (#5520 Series, Hans Rudolph, Kansas City, Mo.) to ensure that the lower airways do not collapse and that the animal does not suffer from hyperventilation.

Once it is determined that baseline values are within the range 0.3-0.9 mL/cm H₂O for compliance and within the range 0.1-0.199 cm H₂O/mL per second for resistance, the pulmonary evaluation is initiated. A Buxco pulmonary measurement computer progam enables the collection and derivation of pulmonary values.

Starting this program initiates the experimental protocol and data collection. The changes in volume over time that occur within the plethysmograph with each breath are measured via a Buxco pressure transducer. By integrating this signal over time, a measurement of flow is calculated for each breath. This signal, together with the pulmonary driving pressure changes, which are collected using a Sensym pressure transducer (#TRD4100), is connected via a Buxco (MAX 2270) preamplifier to a data collection interface (#'s SFT3400 and SFT3813). All other pulmonary parameters are derived from these two inputs.

Baseline values are collected for 5 minutes, after which time the guinea pigs are challenged with ACh. ACh (0.1 mg/mL) is infused intravenously for 1 minute from a syringe pump (sp210iw, World Precision Instruments, Inc., Sarasota, Fla.) at the following doses and prescribed times from the start of the experiment: 1.9 μg/minute at 5 minutes, 3.8 μg/minute at 10 minutes, 7.5 μg/minute at 15 minutes, 15.0 μg/minute at 20 minutes, 30 μg/minute at 25 minutes and 60 μg/minute at 30 minutes. If resistance or compliance has not returned to baseline values at 3 minutes following each ACh dose, the guinea pig's lungs are inflated 3 times with 4 mL of air from a 10 mL calibration syringe. Recorded pulmonary parameters includes respiration frequency (breaths/minute), compliance (mL/cm H₂O) and pulmonary resistance (cm H₂O/mL per second). Once the pulmonary function measurements are completed at minute 35 of this protocol, the guinea pig is removed from the plethysmograph and euthanized by carbon dioxide asphyxiation.

The data are evaluated in one or both of the following ways:

(a) Pulmonary resistance (R_{L}, cm H₂O/mL per second) is calculated from the ratio of “change in pressure” to “the change in flow.” The R_{L} response to ACh (60 μg/min, 1H) is computed for the vehicle and the test compound groups. The mean ACh response in vehicle-treated animals, at each pre-treatment time, is calculated and used to compute % inhibition of ACh response, at the corresponding pre-treatment time, at each test compound dose. Inhibition dose-response curves for ‘R_{L}’ are fitted with a four parameter logistic equation using GraphPad Prism, version 3.00 for Windows (GraphPad Software, San Diego, Calif.) to estimate bronchoprotective ID₅₀ (dose required to inhibit the ACh (60 μg/min) bronchoconstrictor response by 50%). The equation used is as follows:
Y=Min+(Max−Min)/(1+10^{((log ID50-X)*Hillslope)})
where X is the logarithm of dose, Y is the response (% Inhibition of ACh induced increase in R_{L}). Y starts at Min and approaches asymptotically to Max with a sigmoidal shape.

(b) The quantity PD₂, which is defined as the amount of ACh or histamine needed to cause a doubling of the baseline pulmonary resistance, is calculated using the pulmonary resistance values derived from the flow and the pressure over a range of ACh or histamine challenges using the following equation (which is derived from a equation used to calculate PC₂₀ values described in American Thoracic Society. Guidelines for methacholine and exercise challenge testing—1999. Am J Respir Crit Care Med. 161: 309-329 (2000)):

$${PD}_{2} = antilog [ log {C}_{1} + \frac{( log {C}_{2} - log {C}_{1} ) ( 2 {R}_{0} - {R}_{1} )}{{R}_{2} - {R}_{1}} ]$$

where: C₁ is the concentration of ACh or histamine preceding C₂; C₂ is the concentration of ACh or histamine resulting in at least a 2-fold increase in pulmonary resistance (R_{L}); R₀ is the baseline R_{L} value; R₁ is the R_{L} value after C₁; and R₂ is the R_{L} value after C₂. An efficacious dose is defined as a dose that limits the bronchrestriction response to a 50 μg/mL dose of ACh to a doubling of the baseline pulmonary resistance (PD₂₍₅₀₎).

Statistical analysis of the data is performed using a two-tailed Students t-test. A P-value<0.05 is considered significant. Generally, test compounds having a PD₂₍₅₀₎ less than about 200 μg/mL for ACh-induced bronchoconstriction at 1.5 hours post-dose in this assay are preferred. The compound of formula I is expected to have a PD₂₍₅₀₎ less than about 200 μg/mL for ACh-induced bronchoconstriction at 1.5 hours post-dose, when tested in this or a similar assay.

### Assay 4

### Inhalation Guinea Pig Salivation Assay

Guinea pigs (Charles River, Wilmington, Mass.) weighing 200-350 g are acclimated to the in-house guinea pig colony for at least 3 days following arrival. Test compound or vehicle are dosed via inhalation (IH) over a 10 minute time period in a pie shaped dosing chamber (R&S Molds, San Carlos, Calif.). Test solutions are dissolved in sterile water and delivered using a nebulizer filled with 5.0 mL of dosing solution. Guinea pigs are restrained in the inhalation chamber for 30 minutes. During this time, guinea pigs are restricted to an area of approximately 110 sq. cm. This space is adequate for the animals to turn freely, reposition themselves, and allow for grooming. Following 20 minutes of acclimation, guinea pigs are exposed to an aerosol generated from a LS Star Nebulizer Set (Model 22F51, PAR1 Respiratory Equipment, Inc. Midlothian, Va.) driven by house air at a pressure of 22 psi. Upon completion of nebulization, guinea pigs are evaluated at 1.5, 6, 12, 24, 48, or 72 hrs after treatment.

Guinea pigs are anesthetized one hour before testing with an intramuscular (IM) injection of a mixture of ketamine 43.75 mg/kg, xylazine 3.5 mg/kg, and acepromazine 1.05 mg/kg at an 0.88 mL/kg volume. Animals are placed ventral side up on a heated (37° C.) blanket at a 20 degree incline with their head in a downward slope. A 4-ply 2×2 inch gauze pad (Nu-Gauze General-use sponges, Johnson and Johnson, Arlington, Tex.) is inserted in the guinea pig's mouth. Five minutes later, the muscarinic agonist pilocarpine (3.0 mg/kg, SC) is administered and the gauze pad is immediately discarded and replaced by a new pre-weighed gauze pad. Saliva is collected for 10 minutes, at which point the gauze pad is weighed and the difference in weight recorded to determine the amount of accumulated saliva (in mg). The mean amount of saliva collected for animals receiving the vehicle and each dose of test compound is calculated. The vehicle group mean is considered to be 100% salivation. Results are calculated using result means (n=3 or greater). Confidence intervals (95%) are calculated for each dose at each time point using two-way ANOVA. This model is a modified version of the procedure described in Rechter, “Estimation of anticholinergic drug effects in mice by antagonism against pilocarpine-induced salivation” Ata Pharmacol Toxicol 24:243-254 (1996).

The mean weight of saliva in vehicle-treated animals, at each pre-treatment time, is calculated and used to compute % inhibition of salivation, at the corresponding pre-treatment time, at each dose. The inhibition dose-response data are fitted to a four parameter logistic equation using GraphPad Prism, version 3.00 for Windows (GraphPad Software, San Diego, Calif.) to estimate anti-sialagogue ID₅₀ (dose required to inhibit 50% of pilocarpine-evoked salivation). The following equation is used:
Y=Min+(Max−Min)/(1+10^{((log ID50-X)*Hillslope)})
where X is the logarithm of dose, Y is the response (% inhibition of salivation). Y starts at Min and approaches asymptotically to Max with a sigmoidal shape.

The ratio of the anti-sialagogue ID⁵⁰ to bronchoprotective ID⁵⁰ is used to compute the apparent lung selectivity index of the test compound. Generally, compounds having an apparent lung selectivity index greater than about 5 are preferred. The compound of formula I is expected to have an apparent lung-selectivity index greater than about 5, when tested in this or a similar assay.

### ASSAY 5

Methacholine-Induced Depressor Responses in Conscious Guinea Pigs Healthy, adult, male Sprague-Dawley guinea pigs (Harlan, Indianapolis, Ind.), weighing between 200 and 300 g are used in these studies. Under isoflurane anesthesia (to effect), animals are instrumented with common carotid artery and jugular vein catheters (PE-50 tubing). The catheters are exteriorized utilizing a subcutaneous tunnel to the subscapular area. All surgical incisions are sutured with 4-0 Ethicon Silk and the catheters locked with heparin (1000 units/mL). Each animal is administered saline (3 mL, SC) at the end of surgery as well as buprenorphine (0.05 mg/kg, IM). Animals are allowed to recover on a heating pad before being returned to their holding rooms.

Approximately 18 to 20 hours following surgery, the animals are weighed and the carotid artery catheter on each animal is connected to a transducer for recording arterial pressure. Arterial pressure and heart rate are recorded using a Biopac MP-100 Acquisition System. Animals are allowed to acclimate and stabilize for a period of 20 minutes.

Each animal is challenged with MCh (0.3 mg/kg, IV) administered through the jugular venous line and the cardiovascular response is monitored for 10 minutes. The animals are then placed into the whole body dosing chamber, which is connected to a nebulizer containing the test compound or vehicle solution. The solution is nebulized for 10 minutes using a gas mixture of breathable air and 5% carbon dioxide with a flow rate of 3 liters/minute. The animals are then removed from the whole body chamber and returned to their respective cages. At 1.5 and 24 hours post-dosing, the animals are re-challenged with MCh (0.3 mg/kg, IV) and the hemodynamic response is determined. Thereafter, the animals are euthanized with sodium pentobarbital (150 mg/kg, IV).

MCh produces a decrease in mean arterial pressure (MAP) and decrease in heart rate (bradycardia). The peak decrease, from baseline, in MAP (depressor responses) is measured for each MCh challenge (before and after IH dosing). The effects of treatment on the MCh responses are expressed as % inhibition (mean+/−SEM) of the control depressor responses. Two-way ANOVA with the appropriate post-hoc test is used to test the effects of treatment and pre-treatment time. The depressor responses to MCh are expected to be relatively unchanged at 1.5 and 24 hours after inhalation dosing with vehicle.

The ratio of the anti-depressor ID₅₀ to bronchoprotective ID⁵⁰ is used to compute apparent lung-selectivity of the test compound. Generally, compounds having an apparent lung-selectivity index greater than 5 are preferred. The compound of formula I is expected to have an apparent lung-selectivity index greater than 5, when tested in this or a similar assay.

While the present invention has been described with reference to specific aspects or embodiments thereof, it will be understood by those of ordinary skilled in the art that various changes can be made or equivalents can be substituted without departing from the true spirit and scope of the invention. Additionally, to the extent permitted by applicable patent statues and regulations, all publications, patents and patent applications cited herein are hereby incorporated by reference in their entirety to the same extent as if each document had been individually incorporated by reference herein.

What is claimed is:

1. A crystalline naphthalene-1,5-disulfonic acid salt of biphenyl-2-ylcarbamic acid 1-{2-[5-(4-hydroxybenzylamino)pentylcarbamoyl]ethyl}piperidin-4-yl ester or pharmaceutically acceptable solvate thereof.

2. The compound of claim 1, wherein the salt is a mononapadisylate salt.

3. The compound of claim 2, characterized by a powder x-ray diffraction pattern having two or more diffraction peaks at 20 values selected from 5.90±0.2, 9.55±0.2, 10.31±0.2, 12.08±0.2, 13.28±0.2, 15.38±0.2, 15.89±0.2, 17.30±0.2, 18.40±0.2, 19.58±0.2, 21.32±0.2, 22.40±0.2, 23.57±0.2, 24.41±0.2, 25.31±0.2, 26.03±0.2, 26.66±0.2, 27.89±0.2, 28.37±0.2, and 30.83±0.2.

4. The compound of claim 3, wherein the powder x-ray diffraction pattern comprises diffraction peaks at 2θ values of 10.31±0.2, 17.30±0.2, 19.58±0.2, and 24.41±0.2.

5. The compound of claim 2, characterized by a powder x-ray diffraction pattern in which the peak positions are substantially in accordance with the peak positions of the pattern shown in FIG. 1.

6. The compound of claim 2, characterized by a differential scanning calorimetry trace which shows a peak endothermic heat flow at about 176° C.

7. The compound of claim 2, characterized by a differential scanning calorimetry trace substantially in accordance with that shown in FIG. 2.

8. The compound of claim 1, wherein the salt is a heminapadisylate salt.

9. The compound of claim 8, characterized by a powder x-ray diffraction pattern having two or more diffraction peaks at 2θ values selected from 3.56±0.2, 7.19±0.2, 9.71±0.2, 10.76±0.2, 11.81±0.2, 13.11±0.2, 14.65±0.2, 17.06±0.2, 17.84±0.2, 19.61±0.2, 19.94±0.2, 21.23±0.2, 23.30±0.2, and 25.78±0.2.

10. The compound of claim 9, wherein the powder x-ray diffraction pattern comprises diffraction peaks at 2θ values of 3.56±0.2, 10.76±0.2, 17.06±0.2, and 23.30±0.2.

11. The compound of claim 8, characterized by a powder x-ray diffraction pattern in which the peak positions are substantially in accordance with the peak positions of the pattern shown in FIG. 6.

12. The compound of claim 8, characterized by a differential scanning calorimetry trace which shows a peak endothermic heat flow at about 160° C.

13. The compound of claim 8, characterized by a differential scanning calorimetry trace substantially in accordance with that shown in FIG. 7.

14. The compound of claim 1, wherein the salt is in a solvate form.

15. The compound of claim 14, wherein the solvate form is a mononapadisylate ethanolate.

16. The compound of claim 15, characterized by a powder x-ray diffraction pattern having two or more diffraction peaks at 2θ values selected from 8.01±0.2, 9.36±0.2, 11.25±0.2, 12.40±0.2, 14.31±0.2, 15.51±0.2, 16.02±0.2, 16.71±0.2, 18.06±0.2, 18.54±0.2, 19.77±0.2, 20.79±0.2, 21.63±0.2, 21.99±0.2, 22.41±0.2, 23.55±0.2, 24.30±0.2, 25.13±0.2, 26.37±0.2, and 26.91±0.2.

17. The compound of claim 16, wherein the powder x-ray diffraction pattern comprises diffraction peaks at 2θ values of 9.36±0.2, 16.71±0.2, 18.06±0.2, and 18.54±0.2.

18. The compound of claim 15, characterized by a powder x-ray diffraction pattern in which the peak positions are substantially in accordance with the peak positions of the pattern shown in FIG. 11.

19. The compound of claim 15, characterized by a differential scanning calorimetry trace which shows a peak endothermic heat flow at about 167° C.

20. The compound of claim 15, characterized by a differential scanning calorimetry trace substantially in accordance with that shown in FIG. 12.

21. A pharmaceutical composition comprising a pharmaceutically acceptable carrier and a therapeutically effective amount of the compound of claim 1.

22. The composition of claim 21, which further comprises a therapeutically effective amount of an agent selected from β₂ adrenergic receptor agonists, steroidal anti-inflammatory agents, phosphodiesterase-4 inhibitors, and combinations thereof; wherein the compound and the agent are formulated together or separately.

23. The composition of claim 22, which comprises a therapeutically effective amount of a β₂ adrenergic receptor agonist and a steroidal anti-inflammatory agent.

24. The composition of claim 21, wherein the composition is formulated for administration by inhalation.

25. The composition of claim 21, wherein the carrier is an aqueous isotonic saline solution having a pH in the range of from about 4 to 6.

26. A drug delivery device comprising a dry powder inhaler containing a pharmaceutical composition comprising a pharmaceutically acceptable carrier and the compound of claim 1.

27. The compound of claim 1 in micronized form.

28. A pharmaceutical composition comprising a pharmaceutically acceptable carrier and the compound of claim 27.

29. The pharmaceutical composition of claim 28, wherein the carrier is lactose.

30. A process for preparing the compound of claim 1, comprising contacting biphenyl-2-ylcarbamic acid 1-{2-[5-(4-hydroxybenzylaminopentylcarbamoyl]ethyl}piperidin-4-yl ester with 1,5-naphthalenedisulfonic acid.

31. The product prepared by the process of claim 30.

32. A process for purifying biphenyl-2-ylcarbamic acid 1-{2-[5-(4-hydroxybenzylamino)pentylcarbamoyl]ethyl}piperidin-4-yl ester, comprising forming the compound of claim 1.

33. The product prepared by the process of claim 32.

34. A method for antagonizing a muscarinic receptor in a mammal, comprising administering a therapeutically effective amount of the compound of claim 1 to the mammal.

35. A method for treating a pulmonary disorder, comprising administering a therapeutically effective amount of the compound of claim 1 to a patient.

36. A method of producing bronchodilation, comprising administering a bronchodilation-producing amount of the compound of claim 1 to a patient by inhalation.

37. A method of treating chronic obstructive pulmonary disease or asthma, comprising administering a therapeutically effective amount of the compound of claim 1 to a patient.
